SEQUENCE LISTING (89 sequences)

SEQ ID NO: 1 - DNA, 15428 bp, Porcine reproductive and respiratory syndrome virus
cgcccgggcaggtgttggctctatgcctcggcatttgtattgtcaggagctgtgaccattggcacagcccaaaacttgctgcacagaaacacccttctgtgacagcctccttcaggggagcttagggtttgtccctagcaccttgcttccggagttgcactgctttacggtctctccacccctttaaccatgtctgggatacttgatcggtgcacgtgtacccccaatgccagggtgtttgtggcggagggccaagtctactgcacacgatgtctcagtgcacggtctctccttcccctgaacctccaagtttctgagctcggggtgctaggcctgttctacaggcccgaagagccactccggtggacgttgccacgtgcattccccactgttgagtgctcccccgctggggcctgctggctttctgcaatctttccaatcgcacgaatgaccagtggaaacctgaacttccaacaaagaatggtacgggtcgcagctgagatttacagagccggccagctcacccctgcagtcttgaaggctctacaagtttatgaacggggttgccgctggtaccccattgttggacctgtccctggagtggccgttttcgccaattccctacatgtgagtgataaacctttcccgggagcaactcacgtgttgaccaacctgccgctcccgcagagacccaagcctgaggacttttgcccctttgagtgtgctatggctactgtctatgacattggtcatgacgccgtcatgtatgtggccgaagggaaaatctcctgggcccctcgtggcggggatgaagtgaaatttgaagctgtccccggggagttgaagttgattgcgaaccggctccgcacctccttcccgccccaccacgcagtggacatgtctaagttcgccttcacagcccctgggtgtggtgtttctatgcgggtcgaacgccaacacggctgccttcccgctgacactgtccctgaaggcaactgctggtggagcttgtttgacttgctcccactggaagttcaggacaaagaaattcgccatgctaaccaatttggctaccagaccaagcatggtgtctctggcaagtacctacagcggaggctgcaagttaatggtctccgagcagtaactgactcaaacggacctatcgtcgtgcaatacttctccgtcaaggagagttggatccgccatttgaaactggcgggagaacccagctactctgggtttgaggacctcctcagaataagggttgagcctaacacatcgccattggctaacacggaaggaaagattttccggtttggcagtcacaagtggtacggcgctggaaagagagcaagaaaagcacgctcttgtgcgactgctacagtcgctggccgcgctttgtccgttcgtgaaacccggcaggccaaggagcatgaggttgccggcgccgacaaggctgagcacctcaaacactactccccgcctgccgaagggaattgtggttggcactgcatttccgccatcgccaaccggatggtgaattccatctttgaaaccacccttcccgaaagagtgaggcccccagatgactgggctactgacgatgatcttgcgaatgccatccaaatcctcagactccctgcagccttagacaggaacggtgcttgtacaagcgccaagtacgtacttaagctagaaggtgagcattggactgtcactgtgacccctgggatgtccccttctttgctccctcttgaatgtgttcagggctgttgtgagcacaagggcggtcttggttccccagatgcaatcgaggtctctggatttgaccctgcctgccttgactggctggctgaggtgatgcacctgcctagcagtgctatcccagccgctctggccgaaatgtctggcgattccgatcgttcggcttctccggtcaccaccgtgtggactgtttcgcagttctttgcccgtcacagcggagggaatcaccctgaccaagtgcgcttagggaaaattatcagtctctgtcaggtgattgaggactgctgctgttcccagaacaaaaccaaccgggtcaccccggaggaggtcgcagcaaagattgacctgtacctccgtggcgcaacaaatcttgaagaatgcttggccaggcttgagaaagcgcgcccgccgcgcgtaatcgacacctcctttgattgggatgttgtgctccctggggttgaggcggcaacccaaacaaacaagctgccccaggtcaaccagtgtcgtgctctggtccctgttgtgactcaaaagtccttggacaacaactcggtccccctgaccgccttttcactggctaactactactaccgtgcgcaaggtgacgaagttcgtcaccgtgaaagactaaccgccgtgctctccaagttggaagaggttgttcgagaagaatatgggctcatgccaaccgagcctggtccacggcccacactgccacgcgggctcgacgaactcaaagaccagatggaggaggacttgctgagactggctaacgcccaggcgacttcggacatgatggcatgggcagtcgagcaggttgacctaaaaacttgggtcaagaactacccgcggtggacaccaccaccccctccgccaaaagttcagcctcgaaaaacgaagcctgtcaagagcttgccggagagaaagcctgtccccgctccgcgcaggaaggttgggcccgattgtggcagcccggtttcattaggcggcgatgtccctaacagttgggaagatttggctgttagtagccccttggatctcccgaccccacctgagccggcaacactttcaagtgagctagtgattgtgtcctcaccgcaatgcatctttaggccggcgacacccttgagtgagccggctccaattcccgcacctcgcggaactgtgtctcgaccggtgacacccttgagtgagccgatccctgtgcccgcaccgcggcgtaagtttcagcaggtgaaaagattgagttcggcggcggcagtcccactgcaccagaacgagcccctggatttgtctgcttcctcacagactgaatatgaggcctccccctcagcgccgccgcagagcgggggcgttctgggagtagaggggcatgaagctgaggaaaccctgagtgaaatctcggacatgtcgggtaacattaaacctgcgtccgtgtcatcaagcagctccttgtccagcgtggaaatcacacgcccaaaatactcagctcaagccatcatcgactcgggcgggccctgcagtgggcatctccaaggggtaaaggaaacatgccttagtgtcatgcgcgaggcatgtgatgcgactaagcttgatgaccctgctacgcaggaatggctttctcgcatgtgggatcgggtggacatgctgacttggcgcaacacgtctgtttgccaggcgattcgcaccttagatggcaggttaaagttcctcccaaaaatgatactcgagacaccgccgccctatccgtgtgagtttgtgatgatgcctcacacgcctgcaccttccgtaggtgcggagagtgaccttaccattggctcagttgctactgaagatgttccacgcatcctcgagaaaatagaaaatgtcggcgagatggccaaccaggaaccctcggccttctccgaggataaaccggtagatgaccagcttgtcaacgacccccggatatcgtcgcggaggcctgacgagagcacagcagctccgtctgcaggcacaggtggcgccggctcttttaccgatttgccgtcttcagatggcgcggatgcggacgggggggggccgtttcggacggcaaaaagaaaagctgaaaggctctttgatcaactgagccgtcaggtttttgacctcgtctcccatctccctgttttcttctcacgccttttccaccctggcggtggttattctacgggtgattggggttttgcagcttttactctattgtgcctctttttatgttacagttacccagcctttggcattgctcccctcttgggtgtgttttctgggacttctcggcgcgttcgaatgggggtttttggctgctggttggcttttgctgttggtctgttcaagcctgtgtccgacccagtcggcgctgcttgtgagtttgactcgccagagtgtagaaatatccttctttcttttgagcttctcaaaccttgggaccctgttcgcagccttgttgtgggccccgtcggtctcggtcttgccattcttggcaggttactgggcggggcacgctgcatctggcactttttgcttaggcttggcattgttgcagactgtatcttggctggagcttacgtgctttctcaaggcaggtgtaaaaagtgctggggatcttgtataagaactgctcctaatgaggtcgcttttaacgtgttccctttcacacgtgcgaccagatcgtcacttatcgacctgtgcgatcggttttgtgcgccaaaaggaatggaccccatttttctcgccactgggtggcgcggatgctgggccggccgaagccccattgagcaaccctctgaaaaacccatcgcgtttgctcaattggatgaaaagaagattacggctagaactgtggtcgcccagccttatgaccccaaccaagccgtaaagtgcttgcgggtattgcaggcgggtggggcgatggtggctgaggcggtcccaaaagtggtcaaggtttccgctgttccattccgagcccccttctttcccactggagtgaaagttgaccctgattgcagggtcgtagtcgaccctgacactttcactgcagctctccggtctggctactccaccacaaacctcgtccttggtgtaggggactttgcccagctgaatggattaaaaatcaggcaaatttccaagccttcagggggaggcccacatctcatggctgccttgcatgttgcctgctcgatggctctgcacatgcttactgggatttatgtgactgcggtgggttcttgcggcaccggcaccaacgacccgtggtgcgctaacccgtttgccgtccctggctacggacctggctctctgtgcacgtccagattgtgcatttcccaacacggccttaccctgcccttgacagcacttgtggcgggattcggcattcaagaaattgccttggtcgttttgatttttgtttccatcggaggcatggctcataggttgagctgtaaggctgacatgctgtgcattttgcttgcaattgccagctatgtttgggtacctctcacctggttgctttgtgtgtttccttgctggttgcgctgtttttctttgcaccccctcaccatcctatggttggtgtttttcttgatttctgtgaatatgccttcaggaatcttggccatggtgttgttggtttctctttggcttcttggtcgctatactaatgttgctggccttgtcaccccctacgacattcatcattacaccagtggcccccgcggtgttgccgccttggctaccgcaccagatgggacctacttggccgctgtccgccgcgctgcgttgactggccgcaccatgctgtttaccccgtcccagcttgggtctcttcttgagggtgctttcagaactcgaaagccctcactgaacaccgtcaatgtgatcgggtcctccatgggctctggcggggtgtttaccatcgacgggaaggtcaagtgcgtaactgccgcacatgtccttacgggcaattcagctcgggtctccggggtcggcttcaatcaaatgcttgactttgacgtaaagggagatttcgctatagctgattgcccgaattggcaaggggctgcccccaaggcccaattctgcgcggatggatggactggccgtgcctattggctaacatcctctggcgtcgaacccggcgtcattggaaaaggattcgccttctgcttcaccgcatgtggcgattccgggtccccagtgatcaccgaggccggtgagcttgtcggcgttcacacgggatcgaataaacaaggggggggcattgttacgcgcccctcaggccagttttgtaatgtggcacccatcaagctaagcgaattaagtgaattctttgctgggcctaaggtcccgctcggtgatgtgaaggtcggcagccacataattaaagacataagcgaggtgccttcagatctttgtgccttgcttgctgccaaacctgaactggaaggaggcctctccaccgtccaacttctttgtgtgttttttctcctgtggagaatgatgggacatgcctggacgcccttggttgctgtgagtttctttattttgaatgaggttctcccagccgtcctggtccggagtgttttctcctttggaatgtttgtgctctcctggctcacgccatggtctgcgcaaattctgatgatcaggcttctgacagcagcccttaacaggaacagatggtcacttgcctttttcagcctcggtgcagtgaccggttttgtcgcagatcttgcggccactcaggggcatccgttgcaggcagtgatgaatttgagcacctatgcattcctgcctcggatgatggttgtgacctcaccagtcccagtgatcacgtgtggtgtcgtgcacctacttgccatcattttgtacttgtttaagtaccgtggcctgcaccagatccttgttggcgatggagtgttctctgcggctttcttcttgagatactttgccgagggaaagttgagggaaggggtgtcgcaatcctgcggaatgaaccatgagtctctgactggtgccctcgctatgagactcaatgacgaggacttggatttccttatgaaatggactgattttaagtgctttgtttctgcgtccaacatgaggaatgcagcgggtcaatttatcgaggctgcctatgctaaagcacttagagtagaactggcccagttggtgcaggttgataaagttcgaggagttttggccaaacttgaagcttttgctgataccgtggcacctcaactctcgcccggtgacattgttgtcgctctcggccacacgcctgttggcagtatcttcgacctaaaggttggtagcaccaagcataccctccaagccattgagactagagtccttgctgggtccaaaatgaccgtggcgcgcgtcgtcgatccgacccccacgcccccacccgcacccgtgcccattcccctcccaccgaaagttctggagaatggccccaacgcttggggggatgaggaccgtttgaataagaagaagaggcgcaggatggaagccctcggcatctatgttatgggcgggaaaaagtaccagaaattttgggacaagaattccggtgatgtgttttatgaggaggtccataacaacacagatgagtgggagtgtctcagagttggcgaccctgctgactttgaccctgagaagggaactctgtgtggacatgtcaccattgaaaacaaggcttaccatgtttacatctccccatctggtaagaagttcttggtccccgtcaacccagagaatggaagagtccaatgggaagctgcaaagctttccatggagcaggccctaggtatgatgaatgtcgacggcgaactgactgccaaagaactggagaaactgaaaagaataattgacaaactccagggcctgactaaggagcagtgtttaaactgctagccgccagcggcttgacccgctgtggtcgcggcggcttggttgttactgagacagcggtaaaaatagtcaaatttcacaaccggaccttcaccctgggacctgtgaatttaaaagtggccagtgaggttgagctaaaagacgcggttgagcacaaccaacacccggttgcgagaccgatcgatggtggagttgtgctcctgcgttccgcggttccttcgcttatagacgtcttgatctccggtgctgatgcatctcccaagttacttgcccatcatgggccgggaaacactgggatcgatggcacgctctgggattttgagtccgaagccactaaagaggaagtcgcactcagtgcgcaaataatacaggcttgtgacatcaggcgcggcgatgctcctaaaattggtctcccttacaaactgtaccctgttaggggtaaccctgagcgggtgaaaggagttctgcagaatacaaggtttggagacataccttacaaaacccccagtgacactggaagcccagtgcacgcggctgcctgccttacgcccaacgccactccggtgactgacgggcgctccgtcttggccacgaccatgccccccgggtttgagttatatgtaccgaccataccagcgtctgtccttgattaccttgactctaggcctgactgcccaaaacagctgacagagcacggctgcgaagatgccgcactgaaagacctctctaaatatgacttgtccacccaaggctttgttttacctggagtccttcgccttgtgcggaaatacctgtttgcccatgtaggtaagtgcccacccgttcatcggccttctacttaccctgctaagaattctatggctgggataaatgggaacaggttcccaaccaaggacattcagagcgtccctgaaatcgacgttctgtgcgcacaggctgtgcgagaaaactggcaaactgtcaccccttgtactctcaagaaacagtattgcgggaagaagaagactaggaccatactcggcaccaataacttcatcgcactagcccaccgagcagcgttgagtggtgttacccagggtttcatgaaaaaggcgtttaactctcccatcgccctcggaaagaacaagttcaaggagctacagacttcggtcctgggcaggtgccttgaagctgatctcgcatcctgcgatcgatccacgcctgcaattgtccgctggtttgccgccaaccttctttatgaacttgcctgtgctgaagagcatctaccgtcgtacgtgctgaactgctgccacgacttactggtcacgcagtccggcgcggtgactaagagaggtggcctgtcgtctggcgacccgatcacctctgtgtctaacaccatttatagtttggtgatctatgcacagcatatggtgcttagttacttcaagagtggtcacccccatggcctcctgttcttacaagaccagctgaagtttgaggacatgctcaaggtccaacccctgatcgtctattcggacgacctcgtgctgtatgccgagtctcccaccatgccaaactatcactggtgggttgaacatctgaatttgatgctggggtttcagacggacccaaagaagacagcaataacagactcgccatcatttctaggctgtagaataataaatgggcgccagctagtccccaaccgtgacaggatcctcgcggccctcgcctatcacatgaaggcgagcaatgtttctgaatactatgcctcagcggctgcaatactcatggacagctgtgcttgtttggagtatgatcctgaatggtttgaagaacttgtagtcggaatagcgcagtgcgcccgcaaggacggctacagctttcccggcacgccgttcttcatgtccatgtgggagaaactcaggtccaattatgaggggaagaagtcgagagtgtgcgggtactgcggggccccggccccgtacgctactgcctgtggcctcgacgtctgcatttaccacacccacttccaccagcattgtccagtcacaatctggtgtggtcatccagcgggttctggttcttgtagtgagtgcaaatcccctgtagggaaaggcacaagccctttagacgaagtgctggaacaagtcccgtataagcccccacggaccgtcatcatgcatgtggagcagggtctcaccccccttgatccaggtagataccaaactcgccgcggattagtctctgtcaggcgtggaattaggggaaatgaagttgaactaccagacggtgattatgctagcaccgccttgctccctacctgcaaagagatcaacatggtcgctgtcgcttccaatgtattgcgcagcaggttcatcatcggcccacccggtgctgggaaaacatactggctccttcaacaggtccaggatggtgatgttatttacacaccaactcaccagaccatgcttgacatgattagggctttgggaacgtgccggttcaacgtcccggctggcacaacgctgcaattccccgtcccctcccgcaccggtccgtgggttcgcatcctagccggcggttggtgtcctggcaagaattccttcctagatgaagcagcgtattgcaaccaccttgatgttttgaggcttcttagtaaaactaccctcacctgtctaggagacttcaagcaactccacccagtgggttttgactctcattgctatgtttttgacatcatgcctcaaactcaactgaagaccatctggaggtttggacagaatatctgtgatgccattcagccagattacagggacaaactcatgtctatggtcaacacaacccgtgtgacctacgtggaaaaacctgtcaggtatgggcaggtcctcaccccctaccacagggaccgagaggacgacgccatcactattgattccagtcaaggtgccacattcgatgtggttacattgcatttgcccactaaagattcactcaacaggcaaagagcccttgttgccatcaccagggcaagacacgctatctttgtgtatgacccgcacaggcagctgcagggcctgtttgatcttcctgcaaaaggcacacccgtcaacctcgcagtgcaccgcgacgggcagctgatcgtgctggatagaaataacaaagaatgcacggttgctcaggctctaggcaacggggataaatttagggccacagataagcgcgttgtagattctctccgcgccatttgtgctgacctagaagggtcgagctctccgctccccaaggtcgcacacaacttgggattttatttctcacctgatttaacacagtttgctaaactcccagtagaacttgcacctcactggcccgtggtgacaacccagaacaatgaaaagtggccagatcggctggttgccagccttcgccctatccacaaatacagccgcgcgtgcatcggtgccggctatatggtgggcccttcggtgtttctaggcactcctggggtcgtgtcatactatctcacaaaatttgttaagggcgaggctcaattgcttccggagacggttttcagcaccggccgaattgaggtagactgccgggaatatcttgatgatcgggagcgagaagttgctgcgtccctcccacacgccttcattggcgacgtcaaaggcactaccgttggaggatgtcatcatgtcacctccagatacctcccgcgcgtccttcccaaggaatcagttgcggtagtcggggtttcaagccccggaaaagccgcgaaagcattgtgcacactgacagatgtgtacctcccagatcttgaagcctatctccacccggagacccagtccaagtgctggaaaatgatgttggacttcaaagaagttcgactaatggtctggagagacaaaacagcctatttccaacttgaaggtcgctatttcacctggtatcagcttgccagctatgcctcgtacatccgtgttcctgtcaactctacggtgtacttggacccctgcatgggccccgccctttgcaacaggagagtcgtcgggtccacccactggggggctgacctcgcggttaccccttatgattacggcgctaaaatcatcctgtctagcgcgtaccatggtgaaatgccccccggatacaaaattctggcgtgcgcggagttctcgttggatgacccagttaggtacaaacacacttgggggtttgaatcggatacagcgtatctgtatgagttcaccggaaacggtgaggactgggaggattacaatgatgcgtttcgtgcgcgccaggaagggaaaatttacaaggccactgccaccagcttgaagtttcattttcccccgggccctgtcattgaaccaactttaggcctgaattgaaatgaaatggggtccatgcaaagcctttttgacaaaattggccaactttttgtggatgctttcacggagttcttggtgtccattgttgatatcattatatttttggccattttgtttggcttcaccatcgccggttggttggtggtcctttgcatcagattggtttgctccgcgatactccgtacgcgccctgccattcactctgagcaattacagaagatcttatgaggcctttctttcccagtgccaagtggacattcccacctggggaactaaacatcctttggggatgctttggcaccataaggtgtcaaccttgatagatgaaatggtgtcgcgtcgaatgtaccgcatcatggaaaaatcagggcaggctgcctggaaacaggtggtgagcgaggctacactgtctcgcattagtagtttggatgtggtggctcattttcagcatctagccgccattgaagccgagacctgcaaatatttggcctcccggctgcccatgctacacaacctgcgcatgacaggttcaaatgtaaccatagtgtataatagcactttgaatcaggtgtttgctatttttccaacccctggttcccgaccaaagcttcatgattttcagcaatggttaatagctgtacattcctccatattttcctctgttgcagcttcttgtactctttttgttgtgctgtggttgcgggttccaattctacgtactgtttttggtttccgctggttaggggcaatttttcattcgaactcacagtgaattacacggtgtgtccaccttgcctcacccggcaagcagccacagagatctacgaacccggtaggtctctttggtgcaggatagggcatgaccgatgtggggaggatgatcatgacgagctggggtttatggtaccgcctggcctctccagcgaaggccacttgactagtgtttacgcctggttggcgttcttgtccttcagctacacggcccagttccatcccgagatattcgggatagggaatgtgagtcgagtttatgtcgacatcaaacatcaactcatctgcgccgaacatgacgggcagaacgccaccttgtctcgtcatgacaatatttcagccgtgtttcagacctattaccaacatcaagtcgacggcggcaattggtttcacctagaatggcttcgtcccttcttttcctcgtggttggttttaaatgtctcttggtttctcaggcgttcgcctgcaaaccatgtttcagttcgagtcttgcagatattgagaccaacaccaccgcagcggcaggctttgctgtcctccaagacatcagttgccttaggcatcgcgactcggcctctgaggcgattcgcaaaatccctcagtgccgtacggcgatagggacacccgtgtatgttaccatcacagccaatgtgacagatgagaattatttacattcttctgatctcctcatgctttcttcttgccttttctatgcttctgagatgagtgaaaagggatttaaggtggtatttggcaatgtgtcaggcatcgtggctgtgtgtgtcaactttaccagctatgtccaacatgtcaaggagtttacccaacgctccctggtggtcgaccatgtgcggttgctccatttcatgacacctgagaccatgaggtgggcaactgttttagcctgtctttttgccattctgttggcaatttgaatgtttaagtatgttggagaaatgcttgaccgcgggctgttgctcgcgattgctttctttgtggtgtatcgtgccgttctgttttgctgtgctcgccaacgccagcaacagcagcagctcccatctacagctgatttacaacttgacgttatgtgagctgaatggcacagattggctagccaacagatttgattgggcagtggagagttttgtcatttttcccgttttgactcacattgtctcctatggtgccctcaccaccagccattttcttgacacagtcgctttagtcactgtgtctaccgccgggtttgttcacgggcggtatgtcctaagtagcatctacgcggtctgtgccctggctgcgttgacttgctttgtcattaggtttgcaaagaattgcatgtcctggcgctatgcgtgcaccagatataccaactttcttttggacactaagggcagactctatcgttggcggtcgcccgtcatcatagagaaaaggggcaaagttgaggtcgaaggtcatctgatcgacctcaaaagagttgtgcttgatggttccgtggcaacccctataaccagagtttcagcggaacaatggggtcgtccttagatgacttctgtcatgatagcacggctccacaaaaggtgctcttggcgttctctattacctacacgccagtgatgatatatgccctgaaggtgagtcgcggccgactgctagggcttttgcaccttttgatcttcctgaattgtgctttcaccttcgggtacatgactttcacgcactttcagagtacaaataaggtcgcgctcactatgggagcagtagttgcactcctttggggggtgtactcagccatagaaacctggaaattcatcacctccagatgccgtttgtgcttgctaggccgcaagtacattctggcccctgcccaccacgttgaaagtgccgcaggctttcatccgattgcggcaaatgataaccacgcatttgtcgtccggcgtcccggctccactacggtcaacggcacattggtgcccgggttgaaaagcctcgtgttgggtggcagaaaagctgttaagcagggagtggtaaaccttgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccccagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggtgagatttaattggcgagaaccatgcggccgaaattaaaaaaaaaaaaaaaaa

SEQ ID NO: 2 - DNA, 531 bp, Porcine reproductive and respiratory syndrome virus
tgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccatttccctctagcgactgaagatgatgtcagacatcactttacccccagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggtgagatttaattggcgagaaccatgcggccgaaatt

SEQ ID NO: 3 - DNA, 531 bp, Porcine reproductive and respiratory syndrome virus
tgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggtgagatttaattggcgagaaccatgcggccgaaatt

SEQ ID NO: 4 - DNA, 531 bp, Porcine reproductive and respiratory syndrome virus; identical to SEQ ID NO: 3

SEQ ID NO: 5 - DNA, 531 bp, Porcine reproductive and respiratory syndrome virus; identical to SEQ ID NO: 3

SEQ ID NO: 6 - DNA, 531 bp, Porcine reproductive and respiratory syndrome virus; identical to SEQ ID NO: 3

SEQ ID NO: 7 - DNA, 531 bp, Porcine reproductive and respiratory syndrome virus; identical to SEQ ID NO: 3

SEQ ID NO: 8 - DNA, 531 bp, Porcine reproductive and respiratory syndrome virus
tgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggttaacggcactgattgacattgtgcctctaagtcacctattcaattagggcgaccgtgtgggggtgagatttaattggcgagaaccatgcggccgaaatt

SEQ ID NO: 9 - DNA, 531 bp, Porcine reproductive and respiratory syndrome virus
tgtcaaatatgccaaataacaacggcaagcagcagaaaagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttaaattggaagaatgtgtggtgaatggcactgattgacattgtgcctctaagtcacctattcaattagggcgaccgtgtgggggtgagatttaattggcgagaaccatgcggccgaaatt

SEQ ID NO: 10 - DNA, 527 bp, Porcine reproductive and respiratory syndrome virus
aaatatgccaaataataacggcaagcagcagaagagaaagaagggggatggccagccagtcaatcagctgtgccagatgctgggtaagatcatcgctcagcaaaatcagtccagaggcaagggaccgggaaagaaaaataagaagaaaaacccggagaagccccattttcctctagcgactgaagatgatgtcagacatcactttacccccagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggtgagatttaattggcgagaaccatgcggccgaaatt

SEQ ID NO: 11 - DNA, 531 bp, Porcine reproductive and respiratory syndrome virus
  misc_feature (356)..(356): a or g or c or t/u
tgtcaaatatgccaaataacaacggcaagcagctgaagagaaagaagggggatggccagccagtcaatcagctgtgccagatgctgggtaagatcatcgctcagcaaaaccagcccagaggcaagggaccgggaaagaaaaataagaagaaaaacccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgngtcacagcatcaccctcagcatgatgggctggcattnttgaggcatntcagtgtttgaattggaagaatgtgtggtgaatggcactgattgacattgtgcctntaagtcacctattcaattagggcgaccgtgtgggggtgagatttaattggcgagaaccatgcggccgaaatt

SEQ ID NO: 12 - DNA, 527 bp, Porcine reproductive and respiratory syndrome virus
aaatatgccaaataacaccggcaagcagcagaagagaaagaagggggatggccagccagtcaatcagctgtgccagatgctgggtaagatcatcgctcaccaaaaccagtccagaggcaagggaccgggaaagaaaaataagaagaaaaacccggagaagccccatttccctctagcgactgaagatgatgtcagacatcactttacccctagtgagcgtcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatcccagtgtttgaattggaagaatgcgtggtgaatggcactgattgacattgtgcctctaagtcacctattcaattagggcgaccgtgtgggggtaagatttaattggcgagaaccacacggccgaaatt

SEQ ID NO: 13 - DNA, 470 bp, Porcine reproductive and respiratory syndrome virus
tgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgatgatgatgtcagacatcactttacccctagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcaccta

SEQ ID NO: 14 - DNA, 527 bp, Porcine reproductive and respiratory syndrome virus
aaatatgccaaataacaacggcaaacagcagaagaaaaagaagggggatggccagccagtcaatcagctgtgccagatgctgggtaagatcatcgcccagcaaaaccagtccagaggtaagggaccgggaaagaaaaacaagaagaaaaacccggagaagccccattttcctctggcgactgaatatgacgtcagacaccactttacccctagtgagcggcaattgtgcctgtcgtcaatacagactgcctttaatcaaggcgctggtacttgcaccctgtccgattcagggaggataagttacactgtggagtttagtttgcccacgcatcatactgtgcgcctgattcgcgtcacagcatcaccctcagcatgatgggctggcattcttgaggcatctcagtgtttgaattggaagaatgtgtggtgaatggcactgattgatattgtgcctctaagtcacctattcaattagggcgaccgtgtgggggtaagatttaattggcgaaaaccatgcggccgaaatt

SEQ ID NO: 15 - DNA, 484 bp, Porcine reproductive and respiratory syndrome virus
tgtcaaatatgccaaataacaacggcaagcagcagaagagaaagaagggggatggccagccagtcaatcagctgtgccagatgctgggtaagatcatcgcccagcaaaaccagtctagaggcaagggaccgggaaagaaaaataagaagaaaaacccggagaagccccattttcctctagcgactgaagatgatgtcagacatcactttacccctagtgagcggcaattgtgtctgtcgtcaatccagactgcctttaatcaaggcgctgggacttgcaccctgtcagattcagggaggataagttacactgtggagtttagtttgcctacgcatcatactgtgcgcttgatccgcgccacagcatcaccctcagcatgatgggctggcattcttgaggcatccctgtgtttgaattggaagaatgtgtggtgaatggcactgattgacattgtgcctcgaagccacctattcaattagggcga

SEQ ID NO: 16 - DNA, 482 bp, Porcine reproductive and respiratory syndrome virus
tgtcaaatatgccaaataacaacggcaagcagcagaagagaagagaggggatggccagccagtcaatcagctgtgccagatgctgggtaagattatcgctcagcaaaaccagtccagaggcaagggaccgggaaagaaaaataagaagaaaaccccggagaagccccattttcctctagcgactgaagatgatgtcagacatcactttacccctagtgagcggcaattgtgtctgtcgtcaatccagaccgcctttaatcaaggcgctgggacttgcaccctgtcagattcagggagaataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcg

SEQ ID NO: 17 - DNA, 531 bp, Porcine reproductive and respiratory syndrome virus
tgtcaaatatgccaaataacaacggcaagcagcaaaagaaaaagaaggggaatggccagccagtcaaccagctgtgccaaatgctgggcaagatcatcgcccagcagaaccagtccagaggtaagggaccgggaaagaaaattaaaaagaaaaacccggagaagccccattttcctctagcgaccgaagatgacgtcagacatcactttacccctagtgagcggcaattgtgtctgtcgtcaatccagactgcctttaatcaaggcgctggaacttgcaccctgtcggattcagggaggataagttacactgtggagtttagtttgccgacgcatcatactgtgcgcctgattcgcgtcacagcaccaccctcagcgtgatgggctggcattcttgagacatcccagtgttagaattggaagaatgtgtggtgaatggcactgattgacactgtgcctctaagtcacctattcaattagggcgaccgtgtgggggtgagatttaattggcgagaaccatgcggccgaaatt

SEQ ID NO: 18 - DNA, 527 bp, Porcine reproductive and respiratory syndrome virus
aaatatgccaaataacaacggcagacagcaaaagaaaaagaaaggggatggccagccagtcaatcagctgtgtcagatgctgggtaaaatcatcgcccagcaaaaccagtccagaggtaagggaccggggaagaaaaataagaagaaaaacccggaaaagccccattttcctctagcgactgaagatgacgtcagacaccactttacccctagtgagcggcaattgtgtctgtcgtcaatccagacagcctttaatcaaggcgctggaacttgcaccctgtcagattcagggaggataagttacgctgtggagtttagtttgcctacgcatcatactgtgcgcctgattcgcgtcacagcatcaccctcagcatgatgagctggcattcttgagacatcccagtgattgaattggaagaatgtgtggtgaatggcactgattgatattgtgcctctaagtcacctattcaattagggcgaccgtgtgggggtaacatttaattggcgagaaccatgcggccgaaatt

SEQ ID NO: 19 - DNA, 527 bp, Porcine reproductive and respiratory syndrome virus; identical to SEQ ID NO: 18

SEQ ID NO: 20 - DNA, 482 bp, Porcine reproductive and respiratory syndrome virus
tgtcaaatatgccaaataacaacggcaagcagcagaagagaaagaagggggatggccagccagtcaatcagctgtgccagatgctgggtaagatcatcgcccagcaaaaccagtctagaggcaagggaccgggaaagaaaaataagaagaaaaacccggagaagccccattttcctctagcgactgaagatgatgtcagacatcactttacccctagtgagcggcaattgtgtctgtcgtcaatccagactgcctttaatcaaggcgctgggatttgtatcctgtcagattcagggaggataagttacactgtggagtttagtttgcctacgcatcatactgtgcgcttgatccgcgtcacagtatcaccctcagcatgatgggctggcatccttgaggcatcccagtgtttgatttggaagaatgtgtggtgaatggcactgattgacattgtgcctctaagtcacctattcaattagggc

SEQ ID NO: 21 - DNA, 531 bp, Porcine reproductive and respiratory syndrome virus
tgtcaaatatgccaaataacaacggcaagcagcaaaagaaaaagaaggggaatggccagccagtcaatcagctgtgccagatgctgggtaaaatcatcgcccagcaaaaccagtccagaggcaagggaccgggcaagaaaagtaagaagaaaaacccggagaagccccattttcctctagcgaccgaagatgacgtcaggcatcacttcacccctggtgagcggcaattgtgtctgtcgtcgatccagactgcctttaaccagggcgctggaacttgtaccctgtcagattcagggaggataagttacactgtggagtttagtttgccgacgcatcatactgtgcgcctgatccgcgtcacagcatcaccctcagcatgatgggctggcattctttaggcacctcagtgtcagaattggaagaatgtgtggtggatggcactgattgacattgtgcctctaagtcacctattcaattagggcgaccgtgtgggggtaaaatttaattggcgagaaccatgcggccgcaatt

SEQ ID NO: 22 - DNA, 531 bp, Porcine reproductive and respiratory syndrome virus
tgtcaaatatgccaaataacaacggcaagcagcgaaagaaaaagaaggggaatggccagccagtcaatcagctgtgccagatgctgggtaagatcatcgcccaacaaaaccagtccagaggcaagggaccggggaagaaaaataagaagaaaagcccggagaagccccattttcctctagcgaccgaagatgacgtcaggcatcacttcacccctagtgagcggcaattgtgtctgtcgtcgatccagactgcctttaaccagggcgccggaacttgtaccctgtcagattcagggaggataagttacactgtggagtttagtttgccgacgcatcatactgtgcgcctgatccgcgtcacagcatcaccctcagcatgatgggctggtattctttgggcacctcagtgttagaattgggagaatgtgtggtgaatggcactgattgacactgtgcctctaagtcacctattcaattagggcgaccgtgtgggggtgagatttaattggcgagaaccatgcggccgaaatt

SEQ ID NO: 23 - DNA, 454 bp, Porcine reproductive and respiratory syndrome virus
tgtcaaatatgccacataacaacggcaagcagcagaagagaaagaagggggatggccagccagtcaatcagctgtgccagatgctgggtaagatcatcgctcagcaaaaccagtctagaggcaagggaccgggaaagaaatttaagaagaaaaacccggagaagccccattttcctctagcgactgaagatgatgtcagacatcactttacccctagtgagcggcaattgtgtctgtcgtcaatccagactgcctttaatcaaggcgctgggacttgcaccctgtcagattcagggaggataagttacactgtggagtttagtttgcctacgcatcatactgtgcgcttgatccgcgtcacagcatcaccctcagcatgatgggctggcattcttgaggcatcccagtgtttgaattggaagaatgcgtggtgaatggcactgattgacattgt

SEQ ID NO: 24 - DNA, 483 bp, Porcine reproductive and respiratory syndrome virus
tgtcaaatatgccaaataacaacggcaggcagcagaagaaaaagaagggggattgccagccagtcaatcagctgtgccagatgctgggtaagatcatcgcccagcaaaaccagtccagaggcaagggaccgggaaagaaaaataagaagaaaaacccggagaagccccattttcctctagcgactgaagttgatgtcagacatcacttcaccccgagtgagcggcaattgtgtctgtcgtcaatccagactgccttcaatcagggcgctggaacttgtaccctgtcagattcagggaggataagttacactgtggagtttagtttgccgacgcatcatactgtgcgcctgatccgcgttacagcatcaccctcagcatgatgagctggcattcttgaggcatcccagtgtttgaattggaagaatgtgtggtgaatggcactgattgacattgtgcttctaagtcacctattcaattagggcg

SEQ ID NO: 25 - DNA, 531 bp, Porcine reproductive and respiratory syndrome virus
tgtcaaatatgccaaataacaacggcaagcagcagaataaaaagaagggggacggccagccagtcaatcagctgtgccaaatgctgggtaagatcatcgcccagcaaagtcagtccagagttaagggaccgggaaggaaaaataagaagaaaaacccggagaagccccattttcctctggcgactgaagatgacgtcagacaccactttacccccagtgagcggcaattgtgtttgtcgtcaatccagactgcctttaatcaaggcgctggaacttgcatcctgtcagattctgggaggataagttacactgtggagtttagtttgcctacgcatcatactgtgcgcctgatccgcgttacagcaccaccctcagcatgatgagctggcattcttgaggcatcccaatgttcgaattggaagaatgtgtggtgaatggcactgattgatgttgtgcctctaagtcacctattcaattagggcgaccgtgtgggggtataatttaattggcgagaaccacacggccgaaatt

SEQ ID NO: 26 - DNA, 480 bp, Porcine reproductive and respiratory syndrome virus
tgtcaaatatgccaaataacaacggcaaacagcagaagaaaaagaagggggatggccagccagtcaatcagctgtgccagatgctgggtaagatcatcgcccagcaaaaccagtccagaggtaagggaccgggaaagaaaaacaagaagaaaaacccggagaagccccattttcctctggcgactgaatatgacgtcagacaccactttacccctagtgagcggcaattgtgcctgtcgtcaatacagactgcctttaatcaaggcgctggtacttgcaccctgtccgattcagggaggataagttacactgtggagtttagtttgcccacgcatcatactgtgcgcctgattcgcgtcacagcatcaccctcagcatgatgggctggcattcttgaggcatctcagtgtttgaattggaagaatgtgtggtgaatggcactgattgatattgtgcctctaagtcacctattcaattagg

SEQ ID NO: 27 - DNA, 408 bp, Porcine reproductive and respiratory syndrome virus
tgtcaaatatgccaaataacaacggcagacagcagaagaaaaagaagggggatggccagccagtcaatcagctgtgccagatgctgggtaagatcatcgcccagcagagccagtccagaggtaagggaccgggaaagaaaaacaagaagaaaaacccggagaagccccattttcctttagcgactgaagatgacgtcagacattacttcacccctaatgagcggcaattgtgtctgtcgtcaatccatactgccttcaatcaaggcgctggaacttgcaccctgtcagactcagggaggataagttacactgtggagtttagtttgcctacgcagcataccgtgcgcctaatccgcgtcacagcgtcaccctcagcatgatggactggcattcttgagacacctcagt

SEQ ID NO: 28 - DNA, 525 bp, Porcine reproductive and respiratory syndrome virus
aaatatgccaaataacaacggcagacagcagaagaaaaagaagggggatggccagccagtcaatcagctgtgccagatgctgggtaagatcatcgcccagcagagccagtccagaggtaagggaccgggaaagaaaaacaagaagaaaaacccggagaagccccattttcctctagcgactgaagatgacgtcagacatcatttcacccccaatgagcggcaattgtgtctgtcgtcaatccatactgccttcaatcaaggcgctggaacttgcaccctgtcagactcagggaggataagttacactgtggagtttagtttgcctacgcagcataccgtgcgcctaatccgcgtcacagcgtcaccctcagcatgatggactggcattcttgaaacacctcagtgtttgaattggaagatgtgtggtaatggcactgattgatattgtgcctctaagtcacctattcaattagggcgaccgtgtgggggtaggatttaattggcgagaaccacgcggccgaaatt

SEQ ID NO: 29 - DNA, 527 bp, Porcine reproductive and respiratory syndrome virus
aaatatgccaaataacaacggcaaacagcaaaagagaaagacggggaatggccagccagtcaatcagctgtgccagatgctgggtaagattattgcccagcagagtcagtccagaggtaagggaccgggaaataaaaacaagaagaaaaatccggagaagccccattttcctctagcgactgaatatgacgtcagacatcacttcacccctagtgagcggcaattgtgtctgtcgtcaatccagactgcctttaatcaaggcgctggaacttgcaccctgtcagattcagggaggataagttacactgtggagtttagtttgcctacgcatcataccgtgcgccttattcgcgtcacagcatcaccctcagcatgatgagctggcattcttgaggcacctcagtgtttaacttggaagaatgtgtggtgaatggcactgattgatattgtgcctctaagtcacctattcaattagggcgaccgtgtgggggtaggatttaattggcgataaccacgcggccgaaatt

SEQ ID NO: 30 - DNA, 466 bp, Porcine reproductive and respiratory syndrome virus
aaatatgccaaataacaacggcaagcagcagaagaaaaagaaaggggatggccagccagtcaatcagctgtgccagatgctgggtaagatcatcgcccagcaaaaccagtccagaggcaagggaccggggaagaaaaataagaagaaaaacccggagaagccccattttcctctagcgactgaagatgacgtcagacatcacttcacccctagtgagcggcaattgtgtctgtcgtcaatccagactgcctttaatcaaggcgctggaacttgcaccctatcagattcagggagaataagttacgctgtggagtttagtttgcctacgcatcatactgtgcgcctgattcgcgtcacagcatcaccctcagcatgatgagctggcattcttgagacatcccagtgtttgaattggaagaatgtgtggtgaatggcactgattgatattgtgcctctaagtcaccta

SEQ ID NO: 31 - DNA, 452 bp, Porcine reproductive and respiratory syndrome virus
aaatatgccaaataacaacggcaagcagcagaagaaaaagaagggggacggccagccagtcaatcagctgtgccagatgctgggtaaaatcatcgcccagcaaaaccagtccagaggcaagggaccgggaaagaaaaataggaagaaaaacccggagaagccccattttcctctagcgactgaagatgatgtcagacatcactttacccctagtgagcggcaattgtgtctgtcgtcaatccagactgcctttaatcaaggcgctggaacttgtatcctgtcagattcagggaggataagttacactgtggagtttagtttgccgacgcatcataccgtgcgcctgatccgcgtcacagcatcaccctcagcatgatgagctggcattcttgaggcatctcagtgtttgaattggaagaatgtttggtgaatggcactgattgacattgtgc

SEQ ID NO: 32 - DNA, 469 bp, Porcine reproductive and respiratory syndrome virus
tgtcaaatatgccaaataacaacggcaagcagcaaaataagaagaagggggatggccagccagtcaatcagctgtgccagatgctgggtaagattatcgcccagcaaaaccagtccagaggcaagggaccgggaaagaaaaataagaagaaaaacccggagaagccccattttcctctagcgactgaagatgacgtcagacatcactttacccctagtgagcggcaattgtgtctgtcgtcaatccagactgcctttaatcaaggcgctggaacttgtaccctgtcagattcagggaggataagttacactgtggagtttagtttgccgacgcatcatactgtgcgcctgattcgcgtcacagcaccaccctcagcatgatgggctggcattcttgatacacctcagtgttaaaattggaagaatgtgtggtgaatggcactgattgacattgtgcctctaagtcacct

SEQ ID NO: 33 - DNA, 521 bp, Porcine reproductive and respiratory syndrome virus
aaatatgccaaataacgacggcaagcagcaaaagagaaagaaggggaatggccagccagtcaatcagctgtgccaaatgctgggtaagatcatcgcccaacaaaaccagtccagaggcaagggaccggggaagaaaaataagaagaaaaacccggagaagccccattttcctctagcgactgaagatgacgtcaggcatcactttacccctagtgagcggcaattgtgcctgtcgtcgatccagactgcctttaatcagggcgctggaacttgtgccctgtcagattcagggaggataagttacactgtggagtttagtttgccgacgcatcatactgtgcgtctgatccgcgccacagcatcaccctcagcatgatgggctggcattctttggtacctcagtgttagaatcgggagaatgtgtggtgaatggcactgattgacactgtgcctttaagtcacctattcaattagggcgaccgtgtgggggtaaagtttaattggcgagaaccatgcggccg

SEQ ID NO: 34 - DNA, 470 bp, Porcine reproductive and respiratory syndrome virus
tgtcaaatatgccaaataacaacggcaggcagcaaaagaaaaagaagggggatggccagccagtcaaccagctgtgccaaatgctgggtaagatcatcgctcagcaaaaccagtccagaggtaagggaccggggaagaaaaataagaagaaaaacccggaaaagccccattttcctctagcgactgaagatgacgtcagacatcactttacccctagtgagcggcaattgtgtctgtcgtcaatccagactgccttcaatcaaggcgctggaacttgcaccctctcagattcagggaggataagttacactgtggagtttagtttgcctacgcatcatactgtgcgcctgattcgcgttacagcatcaccctcagcatgatgagctggcattcttgagacatcccagtgtttgaattggaagaatgtgtggtgaatggcactgattgatattgtgcctctaagtcaccta

SEQ ID NO: 35 - DNA, 484 bp, Porcine reproductive and respiratory syndrome virus
tgtcaaatatgccaaataacaacggcaagcagcagaagaaaaagaagggggacggccagccagtcaatcagctgtgccaaatgctgggtaagatcatcgcccagcaaagtcagtccagagttaagggaccgggaaggaaaaataagaagaaaaacccggagaagccccattttcctctggcgactgaagatgacgtcagacaccactttacccccagtgagcggcaattgtgtttgtcgtcaatccagactgcctttaatcaaggcgctggaacttgcatcctgtcagattctgggaggataagttacactgtggagtttagtttgcctacgcatcatactgtgcgcctgatccgcgttacagcaccaccctcagcatgatgagctggcattcttgaggcatcccaatgttcgaattggaagaatgtgtggtgaatggcactgattgatgttgtgcctctaagtcacctattcaattagggcga

SEQ ID NO: 36 - DNA, 466 bp, Porcine reproductive and respiratory syndrome virus
aaatatgccaaataacaacggcaagcagcagaagaaaaagaaaggggatggccagccagtcaatcagctgtgccagatgctgggcaggatcatcgcccagcaaaaccagtccagaggtaagggaccggggaagaaaaataagaagaaaaacccggagaagccccattttcctctagcgactgaagatgacgtcagacatcacttcacccctagtgagcggcaattgtgtctgtcgtcaatccagactgcgtttaatcaaggcgctggaacttgtaccctatcagattcagggagaataagttacgctgtggagtttagtttgcctacgcatcatactgtgcgcctgattcgcgtcacagcatcaccctcagcatgatgagctggcattcttgagacatcccagtgtttgaattggaagaatgtgtggtgaatggcactgattgatattgtgcctctaagtcaccta

SEQ ID NO: 37 - DNA, 419 bp, Porcine reproductive and respiratory syndrome virus
gtcaaatatgccaaataacaacggcaagcagcagaagagaaaaaagggggatggccagccagtcaatcagctgtgccagatgctgggcaagatcatcgctcagcaaaaccagttcagaggcaagggaccggggaggaaaaataagaagaaaaacccggagaagccccattttcctctagcgactgaagatgatgtcagacatcactttacccctggtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcggcatgatgggctggcattcttgaggcatctcagtgtttgaattgga

SEQ ID NO: 38 - DNA, 480 bp, Porcine reproductive and respiratory syndrome virus
tgtcaaatatgccaaataacaacggcaggcagcagaagaaaaagaagggggatggccagccagtcaaccagctgtgccagatgctgggtaaaatcatcgcccagcaaaaccagtccagaggcaagggaccgggaaagaaaattaagaagaaaaacccggagaagccccatttccctctatcgattgaatttgttatcagacatcacttcacccctagtgagcggcaattgtgtctgtcgtcaatccagactgcctttaatcaaggcgctggaacttgtaccctgtcagattcagggagaataagttacactgtggagtttagtttgccaacgcatcacactgtgcgcttgatccgcgtcacagcatcaccctcagcatgatgagctggcattcttgaggcatctcagtgtttgaattggaagaatgtatggtgaatggcactgattgacattgtgcttctaagtcacctattcaattagg

SEQ ID NO: 39 - DNA, 151 bp, Porcine reproductive and respiratory syndrome virus
tgggctggcattcttgaggcatctcagtgtttgaattggaagaatgtgtggtgaatggcactgattgacattgtgcctctaagtcacctattcaattagggcgaccgtgtgggggtgagatttaattggcgagaaccatgcggccgaaatt

SEQ ID NO: 40 - DNA, 20 bp, Artificial Sequence
  PRRSV-15308-F Forward primer
gtggtgaatggcactgattg

SEQ ID NO: 41 - DNA, 21 bp, Artificial Sequence
  PRRSV-15303-F Forward Primer
gtgtggtgaatggcactgatt

SEQ ID NO: 42 - DNA, 21 bp, Artificial Sequence
  PRRSV-153309-F3 Forward primer
gtggtgaatggcactgattga

SEQ ID NO: 43 - DNA, 20 bp, Artificial Sequence
  PRRSV-153310-F4 Forward primer
tggtgaatggcactgattga

SEQ ID NO: 44 - DNA, 21 bp, Artificial Sequence
  PRRSV-153308-F5 Forward primer
tgtggtgaatggcactgattg

SEQ ID NO: 45 - DNA, 20 bp, Artificial Sequence
  PRRSV-153309-F6 Forward primer
gtggtgaatggcactgattg

SEQ ID NO: 46 - DNA, 16 bp, Artificial Sequence
  Complement of PRRSV-15360-R reverse primer
gcgaccgtgtgggggt

SEQ ID NO: 47 - DNA, 16 bp, Artificial Sequence
  PRRSV-15360-R reverse primer
acccccacacggtcgc

SEQ ID NO: 48 - DNA, 15 bp, Artificial Sequence
  Complement of PRRSV-15358-R reverse primer
ggcgaccgtgtgggg

SEQ ID NO: 49 - DNA, 15 bp, Artificial Sequence
  PRRSV-15358-R reverse primer
ccccacacggtcgcc

SEQ ID NO: 50 - DNA, 16 bp, Artificial Sequence
  Complement of PRRSV-15357-R reverse primer
agggcgaccgtgtggg

SEQ ID NO: 51 - DNA, 16 bp, Artificial Sequence
  PRRSV-15357-R reverse primer
cccacacggtcgccct

SEQ ID NO: 52 - DNA, 18 bp, Artificial Sequence
  Complement of PRRSV-15356-R reverse primer
attagggcgaccgtgtgg

SEQ ID NO: 53 - DNA, 18 bp, Artificial Sequence
  PRRSV-15356-R reverse primer
ccacacggtcgccctaat

SEQ ID NO: 54 - DNA, 29 bp, Artificial Sequence
  PRRSV-15344-T probe
cctctaagtcacctattcaattagggcga

SEQ ID NO: 55 - DNA, 20 bp, Artificial Sequence
  PRRSV-15344-MGB-1 probe
tcacctattcaattagggcg

SEQ ID NO: 56 - DNA, 20 bp, Artificial Sequence
  PRRSV-15345-MGB-2 probe
cacctattcaattagggcga

SEQ ID NO: 57 - DNA, 18 bp, Artificial Sequence
  PRRSV-15337-MGB-3 probe
cctctaagtcacctattc

SEQ ID NO: 58 - DNA, 19 bp, Artificial Sequence
  PRRSV-15343-MGB-5 probe
agtcacctattcaattagg

SEQ ID NO: 59 - DNA, 20 bp, Artificial Sequence
  PRRSV-15336-MGBR-4 probe
gcctctaagtcacctattca

SEQ ID NO: 60 - DNA, 20 bp, Artificial Sequence
  PRRSV-15343-MGBR-6 probe
agtcacctattcaattaggg

SEQ ID NO: 61 - DNA, 17 bp, Artificial Sequence
  Complement of PRRSV-15409-R reverse primer
gaaccatgcggccgaaa

SEQ ID NO: 62 - DNA, 17 bp, Artificial Sequence
  PRRSV-15409-R reverse primer
tttcggccgcatggttc

SEQ ID NO: 63 - DNA, 16 bp, Artificial Sequence
  Complement of PRRSV-15406-R reverse primer
gagaaccatgcggccg

SEQ ID NO: 64 - DNA, 16 bp, Artificial Sequence
  PRRSV-15406-R reverse primer
cggccgcatggttctc

SEQ ID NO: 65 - DNA, 22 bp, Artificial Sequence
  PRRSV-15302-F forward primer
tgtgtggtgaatggcacttgat

SEQ ID NO: 66 - DNA, 22 bp, Artificial Sequence
  PRRSV-153303-Fa forward primer
gtgtggtgaatggcatttattg

SEQ ID NO: 67 - DNA, 14 bp, Artificial Sequence
  Complement of PRRSV-15360-Ra - reverse primer
cggctgtgtggggg

SEQ ID NO: 68 - DNA, 14 bp, Artificial Sequence
  PRRSV-15360-Fa reverse primer
cccccacacagccg

SEQ ID NO: 69 - DNA, 32 bp, Artificial Sequence
  PRRSV-15332-MGB-1 probe
tgattgacattgtgcctctaagtcacctattc

SEQ ID NO: 70 - DNA, 79 bp, Porcine reproductive and respiratory syndrome virus
agagtgtgtggtgaatggcacttattgacattgtgcctctaagtcacctattcaattagggcggctgtgtgggggtaga

SEQ ID NO: 71 - DNA, 79 bp, Porcine reproductive and respiratory syndrome virus
aaggtgtgtggtgaatggcatttattgacattgtgcctctaagtcacctattcaattagggcggctgtgtgggggtgga

SEQ ID NO: 72 - DNA, 84 bp, Porcine reproductive and respiratory syndrome virus
agaatgttgtggtgaatggcactgattgacacttgtgcctctaagtcacctattcagtttagggccgaccgtgtgggggtagaa

SEQ ID NO: 73 - DNA, 119 bp, Porcine reproductive and respiratory syndrome virus
gctccgatggggaatggccagccagtcaatcaactgtgccagttgctgggtgcaatgataaagtcccagcgccagcaacctaggggaggacaggccaaaaagaaaaagcctgagaagcc

SEQ ID NO: 74 - DNA, 17 bp, Artificial Sequence
  LELY-14997-F forward primer
gtgaatggccgcgattg

SEQ ID NO: 75 - DNA, 18 bp, Artificial Sequence
  Lely-15093-R reverse primer
cggtcacatggttcctgc

SEQ ID NO: 76 - DNA, 28 bp, Artificial Sequence
  Lelystad-15023-T probe
ctctgagtcacctattcaattagggcga

SEQ ID NO: 77 - DNA, 15 bp, Artificial Sequence
  LELY-15005-F2 forward primer
ccgcgattggcgtgt

SEQ ID NO: 78 - DNA, 23 bp, Artificial Sequence
  LELY-15074-R reverse primer
tgattaagtatgacccccatgtg

SEQ ID NO: 79 - DNA, 13 bp, Artificial Sequence
  Euro2-14646-F1 forward primer
atggggaatggcc

SEQ ID NO: 80 - DNA, 14 bp, Artificial Sequence
  Euro2-14646-F2 forward primer
atggggaatggcca

SEQ ID NO: 81 - DNA, 15 bp, Artificial Sequence
  Euro2-14646-F3 forward primer
atggggaatggccag

SEQ ID NO: 82 - DNA, 17 bp, Artificial Sequence
  Euro2-14661-T probe
ccagtcaatcaactgtg

SEQ ID NO: 83 - DNA, 17 bp, Artificial Sequence
  Euro2-14661-BHQ probe
ccagtcaatcaactgtg

SEQ ID NO: 84 - DNA, 11 bp, Artificial Sequence
  Euro2-14661-MGB probe
ccagtcaatca

SEQ ID NO: 85 - DNA, 12 bp, Artificial Sequence
  Euro2-14718-R1 reverse primer
gttgctggcgct

SEQ ID NO: 86 - DNA, 15 bp, Artificial Sequence
  Euro2-14718-R2 reverse primer
ggttgctggcgctgg

SEQ ID NO: 87 - DNA, 16 bp, Artificial Sequence
  Euro2-14718-R3 reverse primer
gttgctggcgctggga

SEQ ID NO: 88 - DNA, 16 bp, Artificial Sequence
  Euro2-14719-R reverse primer
ggttgctggcgctggg

SEQ ID NO: 89 - DNA, 531 bp, Porcine reproductive and respiratory syndrome virus
tgtcaaatatgccaaataacaacggcaagcagcagaagagaaagaagggggatggccagccagtcaatcagctgtgccagatgctgggtaagatcatcgctcagcaaaaccagtccagaggcaagggaccgggaaagaaaaataagaagaaaaacccggagaagccccattttcctctagcgactgaagatgatgtcagacatcactttacccccagtgagcggcaattgtgtctgtcgtcaatccagaccgcctttaatcaaggcgctgggacttgcaccctgtcagattcagggaggataagttacactgtggagtttagtttgcctacgcatcatactgtgcgcctgatccgcgtcacagcatcaccctcagcatgatgggctggcattcttgaggcatctcagtgtttgaattggaagaatgtgtggtgaatggcactgattgacattgtgcctctaagtcacctattcaattagggcgaccgtgtgggggtgagatttaattggcgagaaccatgcggccgaaatt

(19) United States
(12) Patent Application Publication
(10) Pub. No.: US 2005/0170335 A1
(43) Pub. Date: Aug. 4, 2005

(54) Detection of PRRSV

(75) Inventors: Johnny D. Callahan, Severn, MD (US); William Max Nelson, Potomac, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Tetracore, Inc., Gaithersburg, MD (US)

(21) Appl. No.: 10/962,305

(22) Filed: Oct. 8, 2004

Related U.S. Application Data
(60) Provisional application No. 60/510,375, filed on Oct. 9, 2003.

(51) Int. Cl. C12Q 1/70; C12Q 1/68; C12P 19/34

(52) U.S. Cl. 435/5; 435/6; 435/91.2

(57) ABSTRACT

This invention provides compositions and methods for the detection of porcine reproductive and respiratory syndrome viruses (PRRSV). The invention provides oligonucleotides containing sequences complementary to those in ORF 7 and the 3′-UTR (untranslated region) of PRRSV which oligonucleotides may be used to detect the presence of PRRSV sequences, and thus the presence of PRRSV infection, by use of methods provided by the invention. The invention also provides articles of manufacture as well as kits comprising these oligonucleotides which may be used in the detection methods of the invention.

### RELATED APPLICATIONS

This application claims benefit of priority from U.S. Provisional Patent Application 60/510,375, filed Oct. 9, 2003, which is hereby incorporated in its entirety as if fully set forth.

### FIELD OF THE INVENTION

This invention relates to compositions and methods for the detection of porcine reproductive and respiratory syndrome viruses (PRRSV). The invention is practiced with oligonucleotides containing sequences complementary to those in ORF 7 and the 3′-UTR (untranslated region) of PRRSV which oligonucleotides may be used to detect the presence of PRRSV sequences, and thus the presence of PRRSV infection, by use of methods provided by the invention. The invention also relates to articles of manufacture as well as kits comprising these oligonucleotides which may be used in the detection methods of the invention.

### BACKGROUND OF THE INVENTION

Porcine reproductive and respiratory syndrome virus (PRRSV) is responsible for substantial porcine morbidity worldwide and is a source of great economic loss for the pork industry. The virus is a positive-strand RNA virus of the family Arteriviridae. While the origins of the virus in domestic pork industry are unclear, isolates in Europe and North America have been identified as defining two genotypes. The prototypes of European and North American PRRSV are Lelystad virus and VR-2332 (U.S.), respectively. Occurrences of the virus in Canada, China, and Japan appear to be similar to the U.S. prototype while occurrences in Lithuania and other parts of Europe appear similar to the Lelystad prototype. See Plagemann, G W, (2003) Emerg. Infect. Dis. 9(8): 903-908. Detection of viral infection has been by reverse transcription Polymerase Chain Reaction (RT-PCR) mediated methods, but diagnosis by these methods, especially in semen samples, has been difficult due to the inefficiency of RNA extraction and insufficient sensitivity of the tests.

RT-PCR is a laboratory method for the exponential amplification of single stranded RNA, such as that found in a biological sample. The method involves the use of a set of primers and a fluorogenic probe. Real time refers to the ability to monitor the progress of the PCR reaction, most often by fluorometric means as the reaction progresses. Real-time PCR allows quantitative measurements of RNA (or DNA) to be made with much more precision and reproducibility because it relies on threshold cycle (CT) values determined during the exponential phase of PCR rather than endpoint measurements.

One type of real time RT-PCR, using a primer pair and a fluorogenic (dark-hole-quencher) probe (5′-6-FAM/MGB), is based on the hydrolysis of the fluorogenic probe. The probe contains a 5′-fluorophore and a 3′-quencher and anneals to a specific target sequence between the upstream and the downstream primers in a PCR system. The 3′-terminus may be optionally blocked with PO₄, NH₂ or a blocked base. In the presence of the appropriate cycling conditions, the PCR reaction proceeds as the 5′ to 3′-endonuclase activity of the thermal stable polymerase enzyme cleaves the fluorophore from the probe. Because the fluorophore is no longer in close proximity to the quencher its fluorescence becomes detectable. As the concentration of cleaved fluorophore in solution increases, the resultant fluorescent signal is monitored by real-time fluorometric analysis.

Fluorescence values are recorded during every cycle and represent the amount of product amplified to that point in the amplification reaction. The more templates present at the beginning of the reaction, the fewer number of cycles it takes to reach a point in which the fluorescence signal is first recorded as statistically significant above background, which is the definition of the (Ct) values.

Citation of documents herein is not intended as an admission that any is pertinent prior art. All statements as to the date or representation as to the contents of documents is based on the information available to the applicant and does not constitute any admission as to the correctness of the dates or contents of the documents.

### BRIEF SUMMARY OF THE INVENTION

This invention provides compositions and methods relating to the detection of porcine reproductive and respiratory syndrome viruses (PRRSV). The invention thus provides improvements in the field by providing the design of sets of real-time fluorogenic probe based assays to detect PRRSV, including the North American (U.S. and U.S. atypical) and European (European-like or Lelystad) types of PRRSV.

Therefore in one aspect, the invention provides a rapid “one-tube” method that matches the sensitivity and specificity of nested RT-PCR for the detection of PRRSV RNA in a biological sample, such as, but not limited to, semen and blood samples. The “one-tube” embodiment of the invention would thus provide all component reagents needed for real time RT-PCR based detection of PRRSV sequences with the possible exception of the necessary enzymatic activities (reverse transcriptase and DNA polymerase, for example). Thus only the RNA containing sample to be analyzed (optionally with the required enzymatic activities) needs to be added. The method and compositions for use in the methods of the invention may be used to detect PRRSV from a biological sample of any source, including humans and all other animals. It is envisioned that given the ability of infectious diseases to cross the species barrier, there is no expectation that the occurrences of PRRSV sequences are limited to pigs. Preferably, the invention is applied to agricultural animals such as pigs and other livestock. In this embodiment, the invention may be viewed as a veterinary diagnostic product.

The compositions of the invention includes primers and probes for detecting PRRSV, as well as kits containing such primers and probes. One embodiment of such compositions is the “one-tube” detection system described above. These compositions may be used in methods to rapidly identify PRRSV nucleic acids from specimens for diagnosis of PRRSV infection. Using PRRSV specific primers and probes, the methods include amplifying and monitoring the development of specific amplification products using real-time PCR. In a preferred embodiment of the invention, the primers and probes target specific areas of the PRRSV genome known as open reading frame (ORF) 7 and the 3′-UTR. These regions of the viral genome are present in both the Lelystad (European-like) and U.S. prototypes of PRRSV. The specific area targeted by this embodiment of the invention is a 531 base pair (bp) region as identified by the GENBANK reference sequence NC_{—}001961, bases 14881-15411. The particular size of the region may differ depending on the particular PRRSV isolate (and hence genome) of interest, but the identity of ORF 7 and 3′-UTR sequences may be readily identified by comparisons to known PRRSV genome sequences and the nucleocapsid protein N encoded by ORF 7, which immediately precedes the 3′-UTR. As would be apparent to the skilled person, the ORF 7 and 3′-UTR regions may also be readily determined empirically by comparison of a PRRSV genome to the sequence of expressed nucleocapsid protein N, which would define the end of ORF 7 and the start of the 3′-UTR.

In particular embodiments of the invention, the primers and probes are preferably used in one of three assays: 1) a probe hydrolysis based assay that is specific for U.S. PRRSV; 2) a probe hydrolysis based assay specific for both European and Lelystad viruses; and 3) a probe hydrolysis based multiplex assay that utilizes a mixture of the primers utilized in 1) and 2). The latter assay is specific for, and thus able to detect the presence of, sequences from Lelystad virus, European-like PRRSV, U.S. PRRSV, and North American atypical PRRSV.

Therefore, in a related aspect, the invention provides an assay method for detecting the presence or absence of PRRSV in a biological sample from an individual animal. The method to detect PRRSV includes performing at least one cycling step, which includes a nucleic acid amplification step and a hybridization step. The amplifying step includes contacting a sample with at least a pair of PRRSV primers to produce a PRRSV amplification product if a PRRSV nucleic acid molecule is present in the sample, and the hybridizing step includes contacting the sample with at least one PRRSV probe.

In cases utilizing probe hydrolysis based real time RT-PCR as disclosed herein, the at least one PRRSV probe preferably hybridizes to a sequence within the region amplified by a pair of PRRSV primers. This may be the case even where the probe is complementary to (or overlaps with) a portion of one of the two primers (e.g. where the 3′ portion of a probe is complementary to the 3′ portion of a primer as in the case of the use of PRRSV-15303-F, PRRSV-15368-R, and PRRSV-15344-MGB-1 as disclosed herein). PRRSV probe is typically labeled with a donor fluorescent moiety and a second quencher or acceptor fluorescent moiety. The detection methods of the invention further includes detecting the presence or generation of detectable fluorescence, and thus the absence or decrease in fluorescence resonance energy transfer (FRET) between the donor fluorescent moiety and the quencher or acceptor fluorescent moiety in the PRRSV probe. The presence or generation of detectable fluorescence is usually indicative of the presence of PRRSV genetic material in the biological sample, and the absence of detectable fluorescence is usually indicative of the absence of PRRSV in the biological sample.

Detection of fluorescence is preferably the result of amplification using a (thermostable) polymerase enzyme having 5′ to 3′ exonuclease activity which cleaves the donor fluorescence moiety from the probe to result in a detectable signal. The locations of the donor and quencher or acceptor moieties on the probe are preferably such that FRET may occur between the two moieties. The invention contemplates the location of the donor moiety at or near the 5′ end of the probe and the quencher or acceptor moiety at or near the 3′ end of the probe with a separation of from about 14 to about 22 basepairs between the moieties, although other distances, such as from about 6, about 8, about 10, or about 12 basepairs may be used. Preferred distances are about 14, about 16, about 18, about 20, or about 22 basepairs. In an alternative form of such a method, the PRRSV probe can include a nucleic acid sequence that permits secondary structure formation (such as a hairpin) that results in spatial proximity between the donor and the quencher or acceptor fluorescent moiety. Such a method does not require hydrolysis of the probe and has been referred to as the “molecular beacon” approach (see for example, Tyagi S et al. (1996) Molecular beacons: probes that fluoresce upon hybridization. Nat Biotechnol 14, 303-308).

In yet another alternative aspect, the invention provides a method for detecting the presence or absence of PRRSV in a biological sample from an individual as described above except for the use of a pair of probes where one probe contains the donor moiety and the other probe contains the acceptor moiety. Such a method still includes performing at least one cycling step, wherein a cycling step comprises amplification and hybridization. The amplifying step still includes contacting the sample with a pair of PRRSV primers to produce a PRRSV amplification product if a PRRSV nucleic acid molecule is present in the sample. The hybridizing step includes contacting the sample with a pair of probes as described above. The method further includes detecting the presence or absence of fluorescence resonance energy transfer (FRET) between the donor fluorescent moiety and the acceptor fluorescent moiety of the two probes. The presence or absence of FRET is indicative of the presence or absence of PRRSV in the sample. Such a method can further include determining the melting temperature between the PRRSV amplification product and one or both of the probes. The melting temperature can confirm the presence or absence of PRRSV genetic material.

In a further alternative aspect, the invention provides a method for detecting the presence or absence of PRRSV in a biological sample from an individual as described above except for the use of a nucleic acid binding dye in place of any nucleic acid probe. Such a method still includes performing at least one cycling step, wherein a cycling step comprises amplification and a dye-binding step. The amplifying step includes contacting the sample with a pair of PRRSV primers to produce a PRRSV amplification product if a PRRSV nucleic acid molecule is present in the sample. The dye-binding step comprises contacting the PRRSV amplification product with a nucleic acid binding dye. The method further includes detecting the presence or absence of binding of the nucleic acid binding dye to the amplification product. The presence of binding is usually indicative of the presence of PRRSV in the sample, and the absence of binding is usually indicative of the absence of PRRSV in the sample. Non-limiting examples of nucleic acid binding dyes include SybrGreen I®, SybrGold®, and ethidium bromide. Such a method can further include determining the melting temperature between the PRRSV amplification product and the nucleic acid binding dye. The melting temperature can confirm the presence or absence of PRRSV genetic material.

In yet a further aspect, the primers and/or probes of the invention may be used in the PCR based, primer-probe energy transfer (PriProET) system which has been developed for the detection of economically important viruses. Beyond PriProET, the invention may also be used in other fluorimeter-based single and multiplex detection assays of PRRSV as well as other economically important viruses.

A representative donor fluorescent moiety is FAM or 6-FAM, and a representative quencher or acceptor fluorescent moiety is MGB. Other non-limiting examples of a donor moiety include fluorescein, HEX, TET, TAM, ROX, Cy3, Alexa, and Texas Red while non-limiting examples of a quencher or acceptor fluorescent moiety include TAMRA, BHQ (black hole quencher), LC™-RED 640 (LightCycler™-Red 640-N-hydroxysuccinimide ester), LC™-RED 705 (LightCycler™-Red 705-Phosphoramidite), and cyanine dyes such as CY5 and CY5.5. As will be appreciated by a person skilled in the art, any pair of donor and quencher/acceptor moieties may be used as long as they are compatible such that transmission may occur from the donor to the quencher/acceptor. Moreover, pairs of suitable donors and quenchers/acceptors are known in the art and are provided herein. The selection of a pair may be made by any means known in the art and may be confirmed by routine and repetitive testing for energy transfer or quenching of fluorescence.

A pair of PRRSV primers generally includes a first primer and a second primer. The first and second primers can include sequences as disclosed herein as well as additional sequences from within ORF 7 and the 3′-UTR of PRRSV or sequences capable of serving as primers for amplification of sequences from within these regions of PRRSV, such as regions at the 3′ end of ORF 6 (encoding membrane protein M) of PRRSV. Preferably, and in the practice of probe hydrolysis based embodiments of the invention, the primers are no more than about 150, no more than about 100, no more than about 50 nucleotides, no more than about 40, no more than about 30, no more than about 20, or no more than about 10 basepairs from the probe for improved sensitivity in detecting PRRSV sequences.

In yet another aspect, the invention provides primers and probes which may be used in diagnostic screening for multiple isolates of the U.S. type PRRSV, including certain aberrant (mutant) viruses. Given slight variations in the sequences of these various viruses, the invention provides multiple forward primers, reverse primers, and probes which allow RT-PCR amplification of all known US PRRSV isolates. These combinations of primers and probes are optionally used simultaneously in a “one-tube” format as described above, or a single “mastermix” recipe or cocktail, for the detection of US PRRSV genetic material. The invention further provides specific primers and probes for the detection of PRRSV found in Europe and western Asia.

In one aspect of practicing the invention, the detecting step includes exciting the combination of biological sample, primer and probe with a wavelength absorbed by the donor fluorescent moiety and detecting, visualizing and/or measuring fluorescence released from the donor moiety. The amount of detectable fluorescence will depend upon the proximity of the donor moiety to the quencher or acceptor fluorescent moiety. In another aspect, the detecting step is performed after each cycling step, and further, can be performed in real-time. In an alternative aspect, the detecting may comprise quantitating the FRET to the quencher or acceptor fluorescent moiety. The assay methods of the invention are platform independent and work well on at least instrument that support fluorogenic probe hydrolysis assays, including the ABI 7700, the Cepheid Smart Cycler and the Roche Light Cycler.

Generally, the presence of fluorescence in less than about 50 cycles, in less than about 40 cycles, in less than about 30 cycles, or in less than about 20 cycles, indicates the presence of a PRRSV infection in the individual animal from which the sample was obtained. Representative biological samples include oral cavity swabs, cell containing tissues, necropsy tissues, punch biopsies, tonsil scrapings, and bodily fluids such as semen, blood, or serum. Other samples that may be used in the practice of the invention are those that contain mononuclear cells of any kind, but preferably macrophages, such as those present in respiratory tissues and those of alveolar origin.

The methods of the invention can further include preventing amplification of a contaminant nucleic acid. Preventing amplification can include performing the amplification step in the presence of uracil and treating the biological sample with uracil-DNA glycosylase prior to a first amplification step. In addition, the cycling step can be performed on a control sample. A control sample can include a PRRSV nucleic acid molecule. Alternatively, such a control sample can be amplified using a pair of control primers and hybridized using a control probe. The control primers and the control probe are usually other than the PRRSV primers and the probe(s), respectively. A control amplification product is produced if control template is present in the sample, and the control probes hybridize to the control amplification product.

In another aspect of the invention, there are provided articles of manufacture, comprising pairs of PRRSV primers and PRRSV probes with a donor fluorescent moiety with a corresponding quencher or acceptor moiety. The invention also provides articles of manufacture or kits for the practice of methods of the invention in a single tube format which reduces the potential for contamination, simplifies handling of reagents and decreases the likelihood of error, permits rapid (less than about 4 hrs, including RNA extraction), makes use of a ready-to-use RT-PCR “Mastermix” and quality assured, pre-tested and pre-mixed materials; and generates less hazardous waste than traditional gel based RT-PCR methods.

The article of manufacture or kit preferably contains a reagent set comprising an RT-PCR “Mastermix” containing buffers, primers and probe and enzymes ready to load into reaction tubes along with extracted RNA samples. The reagents are optionally shipped as a frozen “Mastermix” with controls for a total reaction volume of 25 μl. The probes in such articles of manufacture or kits can be labeled with a donor fluorescent moiety and with a corresponding quencher or acceptor fluorescent moiety. The articles of manufacture or kits may also optionally include a package label or package insert having instructions thereon for use in a method of the invention.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 is a sequence alignment of position 14881 to 15411 (531 basepairs identified as SEQ ID NO:89) in SEQ ID NO:1 (the sequence of a PRRSV genome identified by the GENBANK reference sequence NC_{—}001961) with other publicly available PRRSV sequences (SEQ ID NOs:2-38). Additional identifiers for each sequence are provided in the Examples below. Positions 14889 to 15260 of SEQ ID NO:1 correspond to ORF 7 and encode the nucleocapsid protein N, while positions 15261-15411 are the 3′-UTR.

FIG. 2 is a representation of the 3′-UTR region of NC_{—}001961 (positions 15261-15411), in lower case letters, with identification of the positions of select primers and probe sequences, in upper case letters, disclosed herein. The complementary (or “comp”) sequences of reverse (or “R”) primers are indicated to avoid the need to show both strands of the 3′-UTR sequence.

FIG. 3 shows curves generated from U.S. PRRSV real-time RT-PCR assay using the Cephid Smart Cycler Instrument. Log 6, 7 and 8 dilutions are shown. The forward primer was PRRSV-15308-F, the reverse primer was PRRSV-15358-R, and the probe was PRRSV-15344-MGB-1.

FIG. 4 shows curves generated from European-Like/Lelystad real-time RT-PCR Assay. Log 3 through log 8 dilutions are shown. The forward primer was PRRSV-14997-F, the reverse primer was PRRSV-15093-R, and the probe was PRRSV-15344-MGB-1.

FIG. 5 is a representation of the 3′-UTR region of NC_{—}001961 (positions 15300-15380), in lower case letters identified as SEQ ID NO:39, with identification of the positions of additional select primers and probe sequences, in upper case letters, disclosed herein. The corresponding sequence from three other PRRSV isolates (1269, 4202, and 18602) are shown. The complementary (or “comp”) sequences of reverse (or “R”) primers are indicated to avoid the need to show both strands of the 3′-UTR sequence.

FIG. 6 shows curves generated from U.S. PRRSV real-time RT-PCR assay using the primers and probes of FIG. 5 in single reactions to detect various PRRSV isolates.

FIG. 7 is a double stranded representation of positions 14640 to 14758 (SEQ ID NO:73) of the ORF7 region of Leylstad virus (GenBank accession number M96262 or M96262.2) with identification of the positions of select primers and probe sequences indicated. The line for the “reverse” primer area of course refers to using the complementary strand to permit PCR amplification in combination with the “sense” (upper) strand sequences of reverse (or “R”) primers are indicated to avoid the need to show both strands of the 3′-UTR sequence.

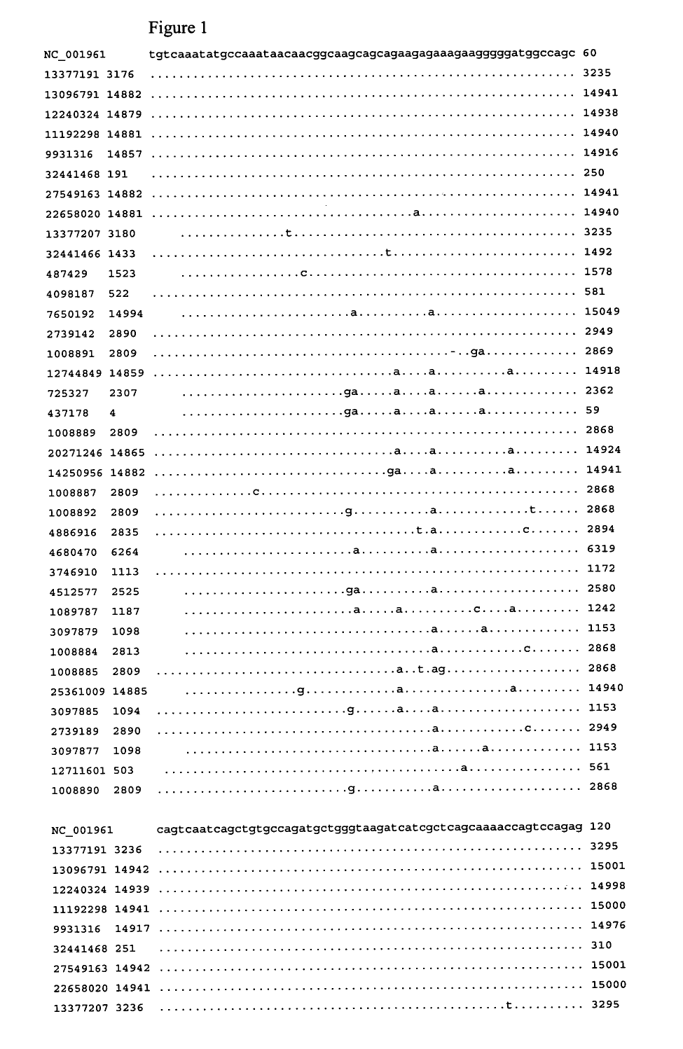

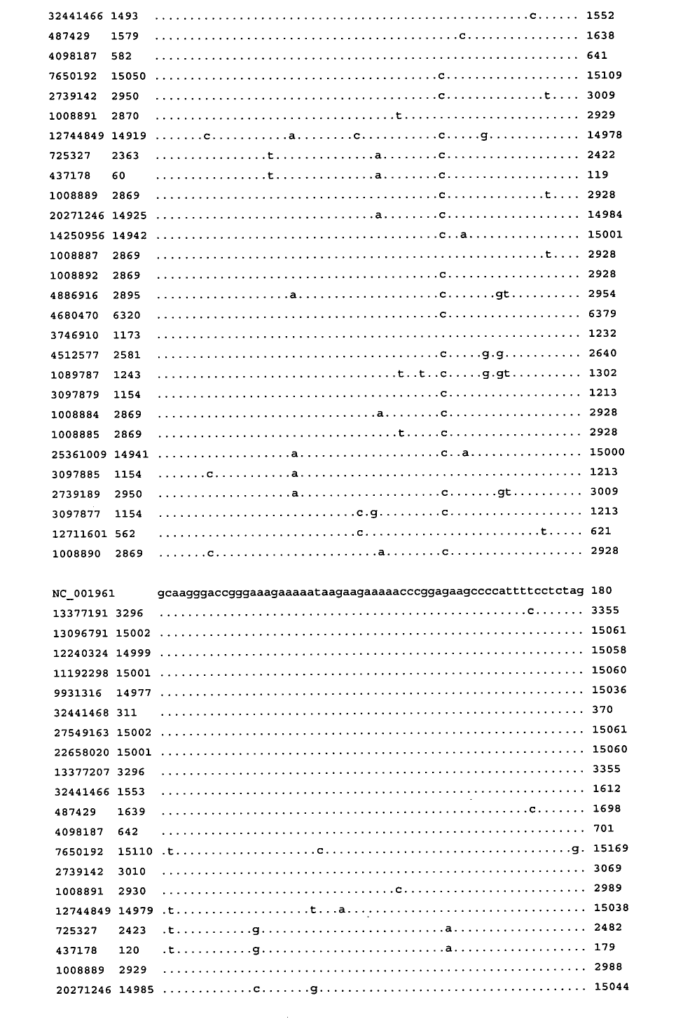

### DETAILED DESCRIPTION

This invention provides a real-time RT-PCR based assay methods for the detection of PRRSV in a biological sample containing PRRSV RNA. Primers and probes for detecting PRRSV infections by amplification of PRRSV nucleic acids and articles of manufacture such as kits containing such primers and probes are provided by the invention. The sensitivity of the real-time RT-PCR assay for detecting PRRSV and the availability of real-time detection of the amplified product, make routine diagnosis of PRRSV infections feasible.

PRRSV and PRRSV Nucleic Acids and Oligonucleotides

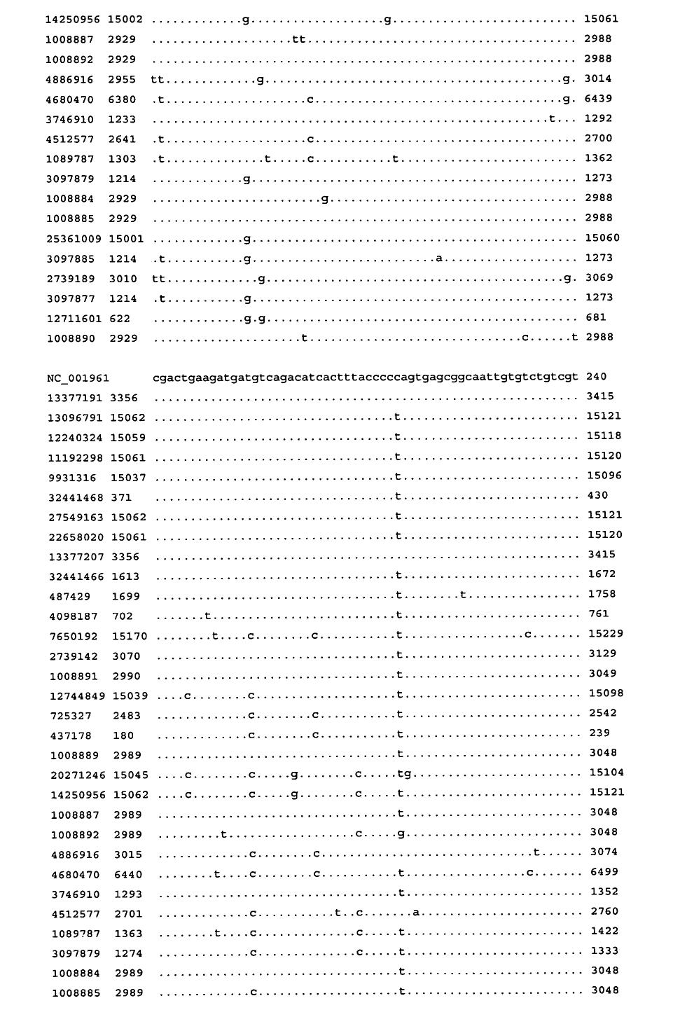

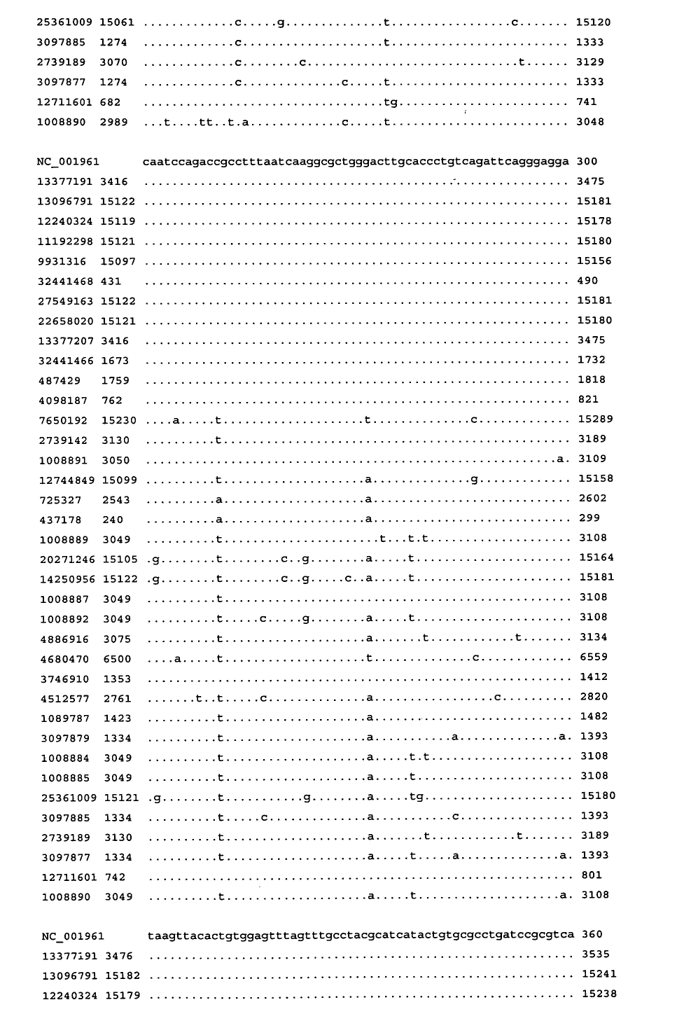

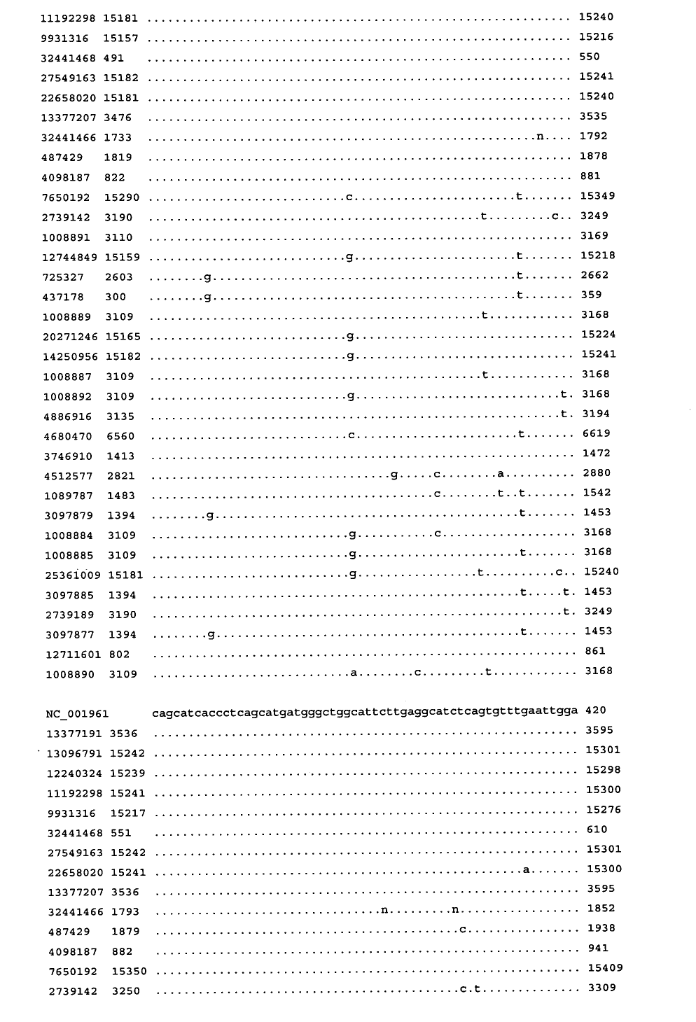

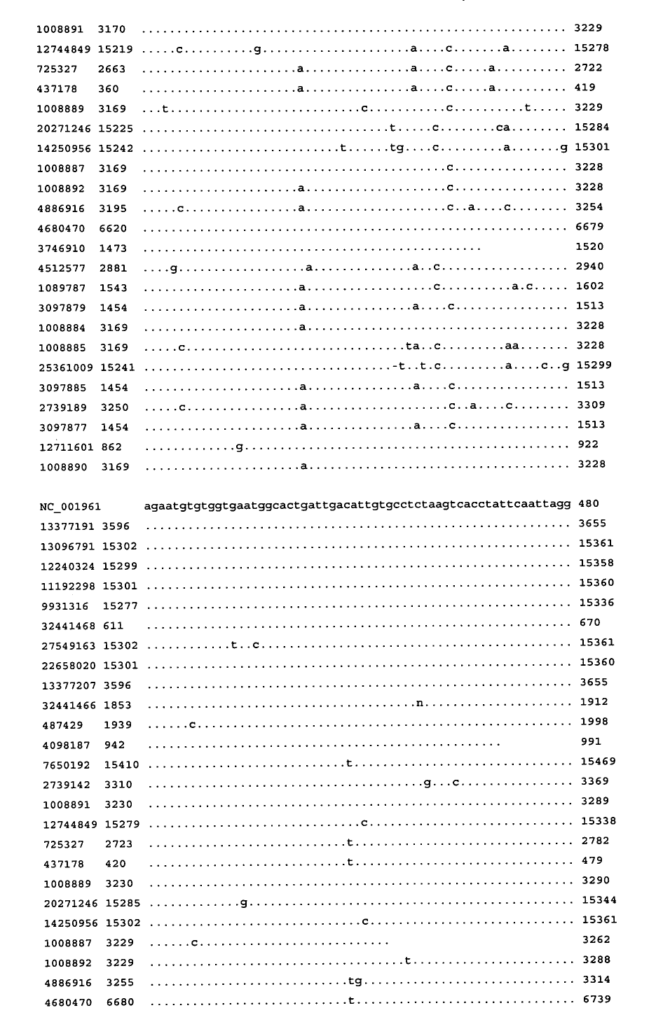

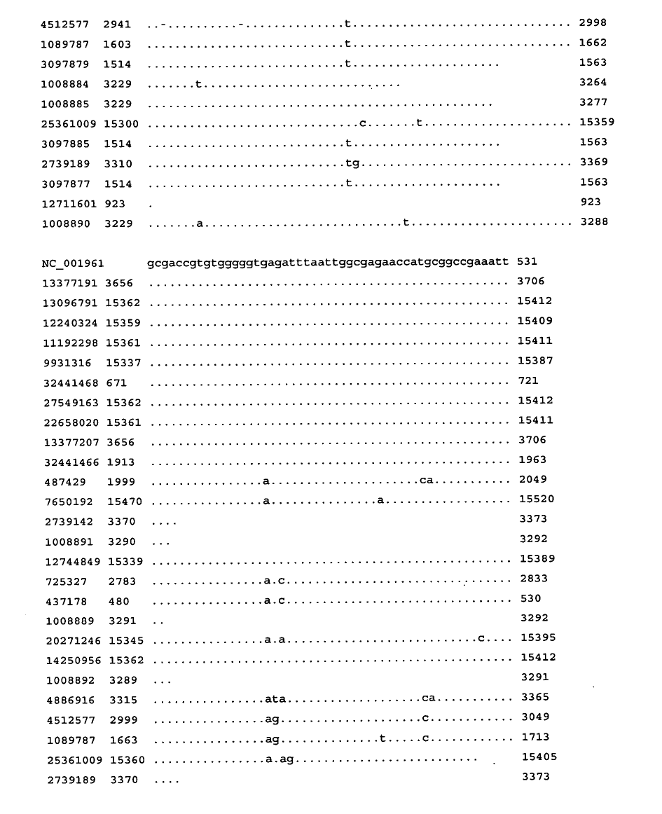

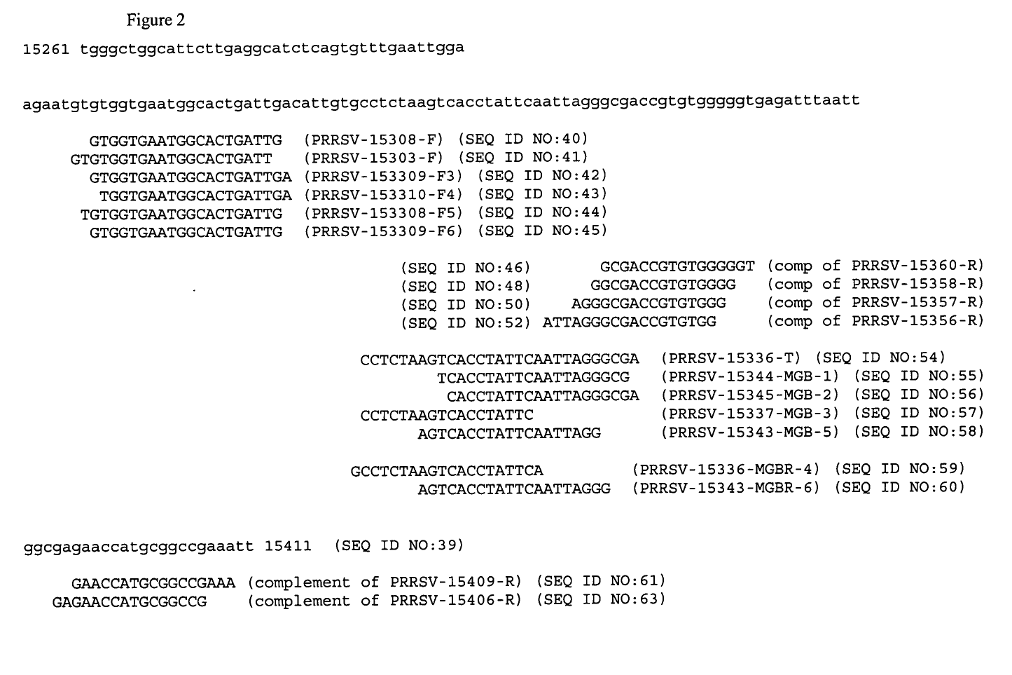

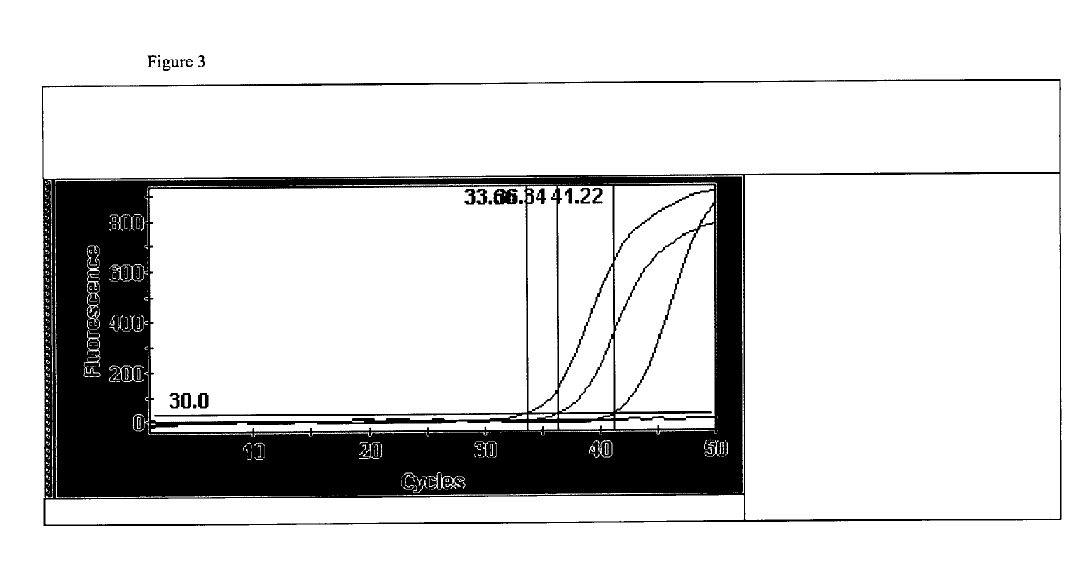

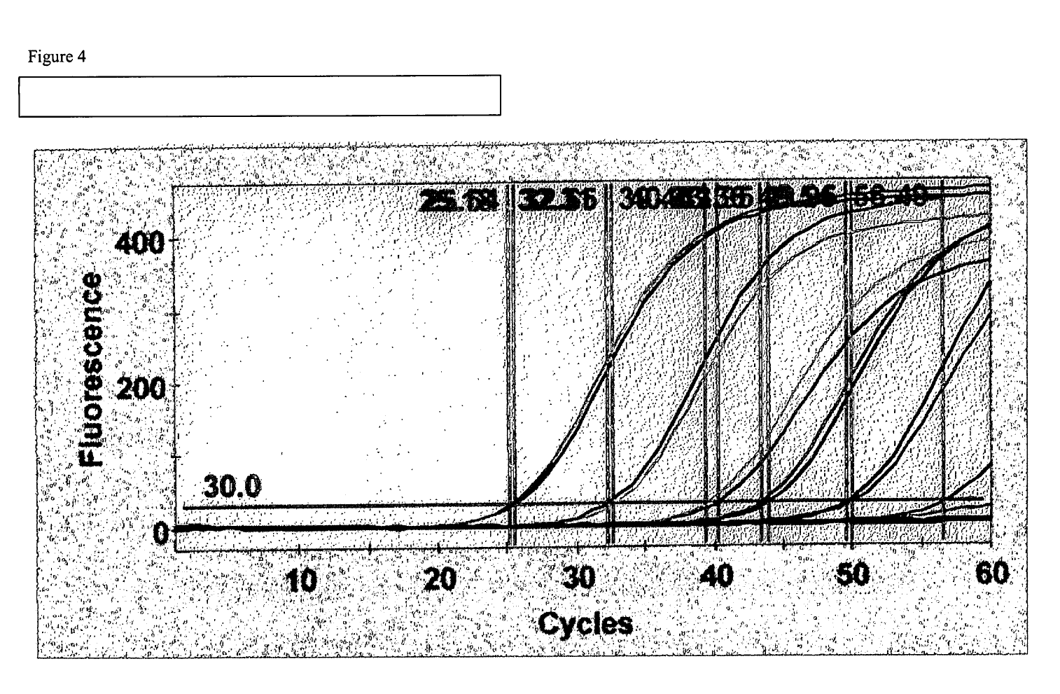

The sequence of one PRRSV genome is provided herein as SEQ ID NO:1. The sequences of a few other isolates have been identified and alignments between them and a portion of SEQ ID NO:1 are shown in FIG. 2. The alignment shows positions within ORF 7 and the 3′-UTR that have been conserved, as well as positions that are variable between different PRRSV genomes. The availability of this information provides the ability to select additional primer sequences and probe sequences for the practice of the invention.

For embodiments of the invention directed to detecting multiple PRRSV genomes with a single primer pair and probe(s), the primers and probes may be selected to be complementary to conserved sequences within ORF 7 and/or the 3′UTR. Alternatively, and for embodiments of the invention directed to detecting a particular PRRSV genome as indicating the presence of a particular isolate, the primers and/or probes may be selected to be complementary to sequences within ORF 7 and/or the 3′UTR that are unique to the particular genome.

As disclosed herein, the invention provides methods to detect PRRSV by amplifying, for example, PRRSV nucleic acid molecules corresponding to all or a part of ORF 7 and/or the 3′-UTR of PRRSV. Given their contiguous structural arrangement, amplification of sequences at the junction between ORF 7 and the 3′-UTR are also contemplated in the practice of the invention. PRRSV nucleic acid molecules other than those exemplified herein (e.g., other than the particular sequences complementary to sequences found in ORF 7 and/or the 3′-UTR) may also be used to detect PRRSV in a sample. Specifically, primers and probes to amplify and detect PRRSV ORF 7 and/or 3′-UTR nucleic acid molecules are provided by the invention. These may include primers complementary to ORF 6 or other region of the PRRSV genome as well as primers and probes that straddle the junction or overlap between ORF 6 and ORF 7. Thus the invention also provides for the amplification of all or part of the sequence found in 14889 to 15260 of SEQ ID NO:1 (corresponding to ORF 7), positions 15261-15411 of SEQ ID NO:1 (corresponding to the 3′-UTR), SEQ ID NO:89 (containing both ORF 7 and the 3′-UTR), or SEQ ID NO:73 (containing positions 14640 to 14758 of the ORF7 region of Leylstad virus). Where all or part of an ORF 7 and/or 3′-UTR is amplified, the size of the amplicon (the region amplified by the primers of the invention and including the length of the primers) is preferably less than about 531 nucleotides, optionally increased by the length of any portion of ORF 6 that is also amplified (in cases wherein a portion of ORF 7 is being amplified). In preferred embodiments, the length of all or part of an ORF 7 and/or 3′-UTR that is amplified may be of a length of less than about 500, less than about 450, less than about 400, less than about 350, less than about 300, less than about 250, less than about 200, less than about 150, less than about 100, less than about 90, less than about 80, less than about 70, or less than about 60 nucleotides. In other preferred embodiments, the amplicon contains only sequences from an ORF 7 or a 3′-UTR.

In embodiments of the invention directed to detection of ORF 7 or 3′-UTR sequences, the invention is preferably practiced based upon a probe hydrolysis method or other real time RT-PCR method. This includes the use of methods comprising a labeled probe that forms a hairpin structure to permit FRET, but this embodiment is less preferred in the practice of the invention.

Primers that amplify a PRRSV nucleic acid molecule, e.g., a portion of ORF 7 and/or the 3′-UTR gene, can be designed by first identifying homology or consensus sequences within a region of the PRRSV genome based upon an alignment of more than one sequence; identifying potential primer and probe sequences, such as those with a higher GC (guanine and cytosine) content or that are likely to have a particular melting temperature (Tₘ,) within the homologous regions; and selecting particular sequences for use as forward and reverse primers as well as probes. In the case of RT-PCR, the selection of primer sequences may also include consideration of the primer used for the reverse transcription step. The selection of primer and probe sequences may be performed with the aid of a computer program such as those available on the internet as NetPrimer and HyTher. Other possibilities include OLIGO from Molecular Biology Insights Inc., Cascade, Colo. Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are about 6 to about 30 nucleotides in length (e.g., about 8, about 10, about 12, about 14, about 16, about 18, about 20, about 22, about 24, about 26, about 28, or about 30 nucleotides in length). “PRRSV primers” as used herein refers to oligonucleotide primers that specifically anneal to PRRSV nucleic acid sequences and initiate nucleic acid synthesis therefrom under appropriate or suitable conditions.

Designing oligonucleotides to be used as hybridization probes can be performed in a manner similar to the design of primers, although the separation between donor and quencher/acceptor moieties in a single probe must not be so great as to prevent fluorescent resonance energy transfer (FRET). Of course, and as would be recognized by the skilled person, the probe(s) for use in the invention are single stranded polynucleotides that may be complementary to either of the strands being amplified in a PCR process. Preferred for use in the invention are oligomers that are complementary to the same strand as the forward primer. In the case of two members of a pair of probes (one containing a donor and one containing a quencher or acceptor moiety), they are preferably designed to anneal to an amplification product within no more than 5 nucleotides of each other (e.g., within no more than 1, 2, 3, or 4 nucleotides of each other) on the same strand such that fluorescent resonance energy transfer (FRET) can occur. It is to be understood, however, that longer separation distances (such as 6 or more nucleotides) are possible if the moieties are appropriately positioned relative to each other (such as by use of a linker) such that FRET can occur. In addition, probes can be designed to hybridize to targets that contain a mutation or polymorphism, thereby allowing differential detection of PRRSV strains based on either absolute hybridization of different probes or optionally via differential melting temperatures between, for example, each probe and each amplification product corresponding to a PRRSV strain to be distinguished. In some embodiments of the invention, the 3′ ends of the probes are blocked to prevent their utilization as a primer in nucleic acid synthesis. Non-limiting examples of blocking groups include PO₄, NH₂ or a blocked base.

As non-limiting examples using appropriate probes, real-time RT-PCR tests for PRRSV have been developed to detect the 3′-UTR of North American PRRSV and European/Lelystad virus, as well as multiplex detection of PRRSV. Multiplex includes the ability to detect more than one PRRSV sequence in the same assay method, such as by the use of more than one primer set with a common labeled probe (e.g. two primer sets, one for U.S. PRRSV and one for Lelystad virus, with a common probe) that is hybridizes to the product amplified by either primer set in the same reaction. Alternatively, it refers to the ability to detect more than one PRRSV sequence in the same assay method by use of more than one primer set with more than one labeled probe (e.g. two primer sets, one for U.S. PRRSV and one for Lelystad virus, with two probes, one for each virus type). As would be understood to the skilled person, the invention may be used to detect the presence of any PRRSV genetic material, and thus the presence of at least one PRRSV isolate or strain. In many embodiments of the invention, the skilled person would find it unnecessary to be aware of the particular isolate, or the number of isolates, being detected because the presence of even one PRRSV isolate or strain is valuable information for the protection and/or treatment of livestock.

Oligonucleotide probes usually have melting temperatures higher than those of the primers used to reduce the occurrence of amplification before annealing of the probe to its cognate sequence. Oligonucleotide probes of the invention are generally of the same lengths as primers of the invention as described above. “PRRSV probes” as used herein refers to oligonucleotide probes that specifically anneal to a PRRSV amplification product.

Constructs of the invention include vectors containing a PRRSV nucleic acid molecule, including a PRRSV 3′-UTR or fragment thereof. The constructs can be used, for example, as a control template nucleic acid. Vectors suitable for use in the present invention are commercially available and/or produced by recombinant DNA technology methods routine in the art. A PRRSV nucleic acid molecule can be obtained, for example, by chemical synthesis, direct cloning from PRRSV, or by PCR amplification. A PRRSV nucleic acid molecule or fragments thereof can be operably linked to a promoter or other regulatory element such as an enhancer sequence, a response element or an inducible element that modulates expression of the PRRSV nucleic acid molecule. As used herein, operably linking refers to connecting a promoter and/or other regulatory elements to a PRRSV nucleic acid molecule in such a way as to permit and/or regulate expression of the PRRSV nucleic acid molecule. A promoter that does not normally direct expression of PRRSV or the PRRSV native promoter can be used to direct transcription of a PRRSV nucleic acid molecule using an appropriate RNA polymerase.

Constructs of the invention containing a PRRSV nucleic acid molecule can be propagated in a host cell, including, but not limited to, prokaryotes and eukaryotes such as yeast, plant and animal cells. A construct of the invention can be introduced into a host cell using any of the techniques commonly known in the art.

Preferred primers and probes of the invention are provided in the following tables.

TABLE 1

PRRSV 3-UTR PROBE HYDROLYSIS ASSAY

| | | |
|---|---|---|
| PRIMERS | SEQUENCE | LENGTH |

| | | | |
|---|---|---|---|
| PRRSV- | GTG GTG AAT GGC | (SEQ ID NO: 40) | 20 |
| 15308-F | ACT GAT TG | | |
| PRRSV- | GTG GTG AAT GGC | (SEQ ID NO: 42) | 21 |
| 153309-F3 | ACT GAT TGA | | |
| PRRSV- | TGG TGA ATG GCA | (SEQ ID NO: 43) | 20 |
| 153310-F4 | CTG ATT GA | | |
| PRRSV- | TGT GGT GAA TGG | (SEQ ID NO: 44) | 21 |
| 153308-F5 | CAC TGA TTG | | |
| PRRSV- | GTG GTG AAT GGC | (SEQ ID NO: 45) | 20 |
| 153309-F6 | ACT GAT TG | | |
| PRRSV- | ACC CCC ACA CGG | (SEQ ID NO: 46) | 16 |
| 15360-R | TCG C | | |
| PRRSV- | CCC CAC ACG GTC | (SEQ ID NO: 48) | 15 |
| 15358-R | GCC | | |
| PRRSV- | CCC ACA CGG TCG | (SEQ ID NO: 50) | 16 |
| 15357-R | CCC T | | |
| PRRSV- | CCA CAC GGT CGC | (SEQ ID NO: 52) | 18 |
| 15356-R | CCT AAT | | |
| PRRSV- | TTT CGG CCG CAT | (SEQ ID NO: 61) | 17 |
| 15409-R | GGT TC | | |
| PRRSV- | CGG CCG CAT GGT | (SEQ ID NO: 63) | 16 |
| 15406-R | TCT C | | |
| LELY- | GTG AAT GGC CGC | (SEQ ID NO: 74) | 17 |
| 14997-F | GAT TG | | |
| Lely- | CGG TCA CAT GGT | (SEQ ID NO: 75) | 18 |
| 15093-R | TCC TGC | | |

PROBES (FAM/TAMRA, MGB Forward and Reverse)

| | | |
|---|---|---|
| (SEQ ID NO: 54) | | |
| PRRSV-15336-T | CCT CTA AGT CAC CTA TTC AAT | 29 |
| | TAG GGC GA | |
| (SEQ ID NO: 76) | | |
| Lelystad-15023-T | CTC TGA GTC ACC TAT TCA ATT AGG | 28 |
| | GCG A | |
| (SEQ ID NO: 55) | | |
| PRRSV-15344-MGB- | TCA CCT ATT CAA TTA GGG CG | 20 |
| 1 | | |
| (SEQ ID NO: 56) | | |
| PRRSV-15345-MGB- | CAC CTA TTC AAT TAG GGC GA | 20 |
| 2 | | |
| (SEQ ID NO: 57) | | |
| PRRSV-15337-MGB- | CCT CTA AGT CAC CTA TTC | 18 |
| 3 | | |
| (SEQ ID NO: 58) | | |
| PRRSV-15343-MGB- | AGT CAC CTA TTC AAT TAG G | 19 |
| 5 | | |
| (SEQ ID NO: 59) | | |
| PRRSV-15336- | TGA ATA GGT GAC TTA GAG GC | 20 |
| MGBR-4 | | |
| (SEQ ID NO: 60) | | |
| PRRSV-15343- | CCC TAA TTG AAT AGG TGA CT | 20 |
| MGBR-6 | | |

One Final Configuration for amplification of the
3′-UTR

| | | |
|---|---|---|
| (SEQ ID NO: 41) | | |
| PRRSV-15303-F | GTG TGG TGA ATG GCA CTG ATT | 21 |
| (SEQ ID NO: 48) | | |
| PRRSV-15358-R | CCC CAC ACG GTC GCC | 15 |
| (SEQ ID NO: 77) | | |
| LELY-15005-F2 | CCG CGA TTG GCG TGT | 15 |
| (SEQ ID NO: 78) | | |
| LELY-15074-R | TGA TTA AGT ATG ACC CCC ATG | 23 |
| | TG | |
| (SEQ ID NO: 55) | | |
| PRRSV-15344-MGB- | TCA CCT ATT CAA TTA GGG CG | 20 |
| 1 | | |

The use of “F” and “R” denote “forward” and “reverse” primers relative to PCR amplification. “PRRSV” refers to both North American and atypical North American PRRSV sequences. “LELY” refers to the Lelystad strain of PRRSV. The numeric designations may be related to positions in SEQ ID NO:1.

One particularly preferred embodiment of the invention is set forth in the “final configuration” which presents a pair of PRRSV specific primers and a pair of Lelystad (“LELY”) specific primers where both pairs can function with the indicated probe either alone or in a multiplex reaction. Stated differently, the PRRSV primers and the PRRSV-15344-MGB-1 probe can detect the presence of North American (and atypical) PRRSV in methods as disclosed herein while the LELY primers with the same probe can detect Lelystad (and European-like) virus in methods as disclosed herein. All four primers and the probe may be present in the same reaction to detect either, or both, North American PRRSV and Lelystad virus in a multiplex format.

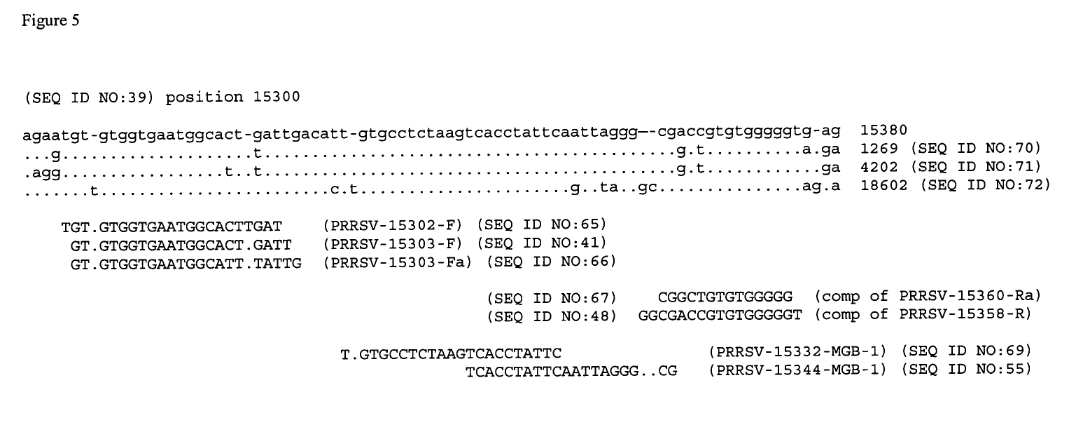

In another preferred embodiment, the primers and probes of FIG. 5 are used to detect North American PRRSV. Combinations of functional sets of primers and probes may be used in separate reactions or be present in the same reaction to detect various isolates of North American PRRSV.

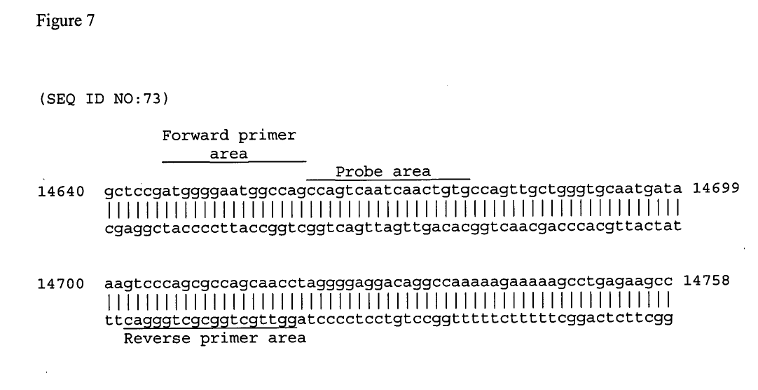

In a further preferred embodiment, the following primers and probe may be used to detect various isolates of European and Western Asia PRRSV. The sequences of these primers and probes correspond to those in the ORF7 region as shown in FIG. 7.

| | | |
|---|---|---|
| Forward Primers | | |
| Euro2-14646-F1 | ATG GGG AAT GGC C | (SEQ ID NO: 79) |
| Euro2-14646-F2 | ATG GGG AAT GGC CA | (SEQ ID NO: 80) |
| *Euro2-14646-F3 | ATG GGG AAT GGC | (SEQ ID NO: 81) |
| | CAG | |
| Probes | | |
| Euro2-14661-T | CCAGTCAATCAACTGTG | (SEQ ID NO: 82) |
| | (FAM/TAMRA) | |
| Euro2-14661-BHQ | CCAGTCAATCAACTGTG | (SEQ ID NO: 83) |
| | (FAM/DHQ) no MGB | |
| *Euro2-14661-MGB | CCAGTCAATCA (MGB) | (SEQ ID NO: 84) |
| Reverse primers | | |
| Euro2-14718-R1 | GTT GCT GGC GCT | (SEQ ID NO: 85) |
| Euro2-14718-R2 | GGT TGC TGG CGC | (SEQ ID NO: 86) |
| | TGG | |
| *Euro2-14718-R3 | GTT GCT GGC GCT | (SEQ ID NO: 87) |
| | GGG A | |
| Euro2-14719-R | GGT TGC TGG CGC | (SEQ ID NO: 88) |
| | TGG G | |

The primers and probe indicated with an asterisk represent a preferred combination for use, alone or in combination with primers and/or probes for other PRRSV genetic material, in the pratice of the invention.

As would be readily apparent to the skilled person in the art, the possibility exists for slight extensions or truncations of each primer and probe sequence disclosed herein without affecting the functionality of the primers and probes in an RT-PCR based reaction. Therefore, the invention also contemplates the use of slightly longer or slightly shorter versions of the primer and probe sequences disclosed herein where the slight increase or decrease in length may be due to extensions and truncations at one or both ends (i.e. both 5′ and 3′ ends) of a sequence. Of course the extensions and truncations are not by addition or deletion of random nucleotides, but rather they are made with reference to PRRSV sequences to avoid deleterious effects on the functionality of the primers and probes in use in the practice of the instant invention. By “slightly longer or slightly shorter”, it is meant an increase or decrease of about 1-2 nucleotides relative to each end of a sequence, or a change up to about 10-15% of the length of the sequence. In the case of “slightly longer”, the primers and probes may of course be lengthened further, and up to the longest lengths disclosed herein for use in the practice of the invention.

RT-PCR

Conventional PCR techniques are disclosed in U.S. Pat. Nos. 4,683,202, 4,683,195, 4,800,159, and 4,965,188. Briefly, PCR typically employs two oligonucleotide primers that bind to a selected nucleic acid template (e.g., DNA or RNA) and its complement. Primers for use in the present invention include oligonucleotides capable of serving as the start of nucleic acid synthesis within a PRRSV nucleic acid sequence. The nucleic acid synthesis is usually mediated by a thermostable polymerase activity. A primer may be produced synthetically via a DNA synthesizer. A primer is preferably single-stranded for maximum efficiency in amplification, but a primer may also be used after denaturation, such as by heating, to separate the two strands.

In RT-PCR, a starting RNA template, such as mRNA, is first converted to DNA by use of a reverse transcriptase activity.

The term “thermostable polymerase” refers to a polymerase enzyme that is heat stable and thus does not irreversibly denature when subjected to the elevated temperatures for the time necessary to effect denaturation of double-stranded template nucleic acids. The polymerase activity catalyzes the formation of primer extension products complementary to a template while a 5′ to 3′ exonuclease activity may also be present. Generally, nucleic acid synthesis is initiated at the 3′ end of each primer and proceeds in the 5′ to 3′ direction along the template strand. Thermostable polymerases isolated from many organisms may be used in the practice of the invention. Polymerases that are not thermostable also can be employed in PCR if it is replenished during PCR.

PCR assays can be used with unpurified nucleic acid templates or where the template may be a minor fraction of a complex mixture, such as PRRSV nucleic acids contained in infected cells. However, and as discussed below, the invention provides improved methods of extracting RNA for use as the starting material in the methods of the invention.

Samples containing PRRSV genetic material for use as a template may be obtained from a variety of biological fluids and tissues. The template is combined with the oligonucleotide primers and with other PCR reagents under reaction conditions that support primer extension. For example, chain extension reactions generally include 50 mM KCl, 10 mM Tris-HCl (pH 8.3), 1.5 mM MgCl₂, 0.001% (w/v) gelatin, 0.5-1.0 μg denatured template DNA, 50 pmoles of each oligonucleotide primer, 2.5 U of Taq polymerase, and 10% DMSO. The reactions usually contain 150 to 320 μ.M each of DATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce a quantity of amplification products corresponding to the target PRRSV nucleic acid molecule. The limiting factors in the reaction are the amounts of primers, thermostable enzyme, and nucleoside triphosphates present in the reaction. The cycling steps (i.e., amplification and hybridization) are preferably repeated at least once. The number of cycling steps will depend on a variety of factors, including the nature of the sample. As a non-limiting example, if the sample is a complex mixture of nucleic acids, more cycling steps may be required to amplify the target sequence sufficient for detection. Generally, the cycling steps are repeated at least about 10 or about 20 times, but may be repeated as many as about 40 or more, about 60 or more, or even about 100 or more times.

Fluorescent Resonance Energy Transfer (FRET)

FRET technology is discussed in U.S. Pat. Nos. 4,996,143, 5,565,322, 5,849,489, and 6,162,603. FRET is based on the phenomenon that when a donor and a corresponding acceptor moiety are positioned within a certain distance of each other, energy transfer takes place between the two moieties. The transferred can be visualized or otherwise detected and/or quantitated. Alternatively, the transfer can be a quenching of the fluorescence of the donor such that interruption of the transfer results in the emission of detectable fluorescence.

As used herein with respect to donor and corresponding quencher or acceptor moieties, “corresponding” refers to a quencher or acceptor moiety having an emission spectrum that overlaps the excitation spectrum of the donor fluorescent moiety. The wavelength maximum of the emission spectrum of the quencher or acceptor moiety preferably should be at least 100 nm greater than the wavelength maximum of the excitation spectrum of the donor fluorescent moiety. This results in efficient non-radiative energy transfer between the two moieties.

Fluorescent donor and corresponding quencher or acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding quencher or acceptor moiety. A corresponding quencher or acceptor moiety can be chosen that has a high extinction coefficient, a high quantum yield, a good overlap of its excitation with the emission of the donor fluorescent moiety, and emission in the red part of the visible spectrum (>600 nm).

Representative, and non-limiting, donor fluorescent moieties that can be used with various acceptor fluorescent moieties in FRET technology include fluorescein, Lucifer Yellow, B-pliycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimidyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC™-RED 640 (LightCycler™-Red 640-N-hydroxysuccinimide ester), LC™-RED 705 (LightCycler™-Red 705-Phosphoramidite), cyanine dyes such as CY5 and CY5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and quencher or acceptor moieties can be attached to the appropriate probe oligonucleotide via a linker. The length of each linker arm can be important, as the linker arms will affect the distance between the donor and the quencher or acceptor moieties. The length of a linker for the purpose of the present invention is the distance in Angstroms (Å) from the nucleotide base to the fluorescent moiety. In general, a linker is from about 10 to about 25 Å. A variety of linkers are known in the field and may be used in the present invention.

Detection of PRRSV

The present invention is preferably embodied in a single tube method that is easy to perform and provides results in less than 4 hours (including RNA extraction). In one embodiment, the test is performed in four easy steps: 1) addition of enzymes to a “Mastermix” containing the necessary reagents; 2) addition of about 4 μl of sample (extracted RNA) or control to each tube or well; 3) briefly centrifuge the reaction tubes or wells to concentrate the solutions; and 4) place the reactions into a PCR instrument with the ability to detect real-time fluorescence and run a recommended PCT protocol.

The specificity of the instant invention was demonstrated by each of two RT-PCR diagnostic reagent sets (each having one primer pair) for US (North American) and European-like/Lelystad virus) having 100% specificity in single format based on the panel selected. The panel included 12 European-like PRRSV isolates from a diverse geographic area in the U.S., Lelystad virus, and over 50 diverse U.S. PRRSV isolates (including atypical PRRSV). The assay failed to amplify RNA extracted from other arteriviruses (EAV and LDV) as well as non-related swine viruses.

The sensitivity of the instant invention included where the PRRSV diagnostic reagent sets (each with one primer pair) matched the sensitivity and specificity of nested RT-PCR assays for both North American and European-like PRRSV. The North American type diagnostic reagent set detected up to a 10⁻⁸ dilution of a SDSU23983 stock virus while the European-Like type diagnostic reagent set detected up to a 10⁻⁷ dilution of Lelystad virus. Both of these results matching the sensitivity of nested RT-PCR, which has a reported sensitivity of approximately 10 virions/ml.

The invention provides methods for detecting the presence or absence of PRRSV in a biological sample from an individual. The methods include performing at least one cycling step that includes amplifying and hybridizing where the amplification step includes contacting the biological sample with a pair of PRRSV primers to produce a PRRSV amplification product if a PRRSV nucleic acid molecule is present in the sample. Each of the primers anneals to a target within or adjacent to a PRRSV nucleic acid molecule such that at least a portion of the amplification product contains nucleic acid sequence corresponding to PRRSV. More importantly, the amplification product contains the nucleic acid sequences that are complementary to PRRSV probes. A hybridizing step includes contacting the sample with one or more PRRSV probes. Multiple cycling steps can be performed, preferably in a thermocycler.

As used herein, “amplifying” refers to the process of synthesizing nucleic acid molecules that are complementary to one or both strands of a template nucleic acid, such as PRRSV nucleic acid molecules). Amplifying a nucleic acid molecule typically includes denaturing the template nucleic acid, annealing primers to the template nucleic acid at a temperature that is below the melting temperatures of the primers, and enzymatically elongating from the primers to generate an amplification product. The denaturing, annealing and elongating steps each can be performed once per cycle. Generally, however, the denaturing, annealing and elongating steps are performed in multiple cycles such that the amount of amplification product is increasing, often times exponentially, although exponential amplification is not required by the present methods. Amplification typically requires the presence of deoxyribonucleoside triphosphates, a DNA (thermostable) polymerase enzyme and an appropriate buffer and/or co-factors for optimal activity of the polymerase enzyme.

If amplification of PRRSV nucleic acid occurs and an amplification product is produced, the step of hybridizing results in the annealing of one or more probe molecules to the product via base pair complementarity. Hybridization conditions typically include a temperature that is below the melting temperature of the probes from the amplification product but that avoids non-specific hybridization of the probes.

In the case of probe hydrolysis to generate a detectable signal, the 5′ to 3′ exonuclease activity of a (thermostable) DNA polymerase is used to release a fluorescent moiety from being quenched or subdued by a quencher or acceptor present on the same probe molecule.

In the case of a pair of probes, each containing one of a donor and quencher or acceptor moieties, the presence of FRET indicates the presence of PRRSV in the biological sample, and the absence of FRET indicates the absence of PRRSV in the biological sample.

Within each thermocycler run, control samples can be cycled as well. Positive control samples can amplify control nucleic acid template (preferably one other than PRRSV) using, as a non-limiting example, control primers and control probes. Positive control samples can also amplify, as a non-limiting example, a plasmid construct containing PRRSV nucleic acid molecules. Such a plasmid control can be amplified internally (such as within each biological sample) or in separate samples run side-by-side with the test samples. Each thermocycler run also should include a negative control that, for example, lacks PRRSV template DNA. Such controls are indicators of the success or failure of the amplification, hybridization, and/or detection steps. Therefore, control reactions can readily determine, for example, the ability of primers to anneal with sequence-specificity and to initiate elongation, as well as the ability of probes to hybridize with sequence-specificity.

The methods of the invention include steps to avoid contamination. As a non-limiting example, an enzymatic method utilizing uracil-DNA glycosylase is described in U.S. Pat. Nos. 5,035,996, 5,683,896 and 5,945,313 to reduce or eliminate contamination between one thermocycler run and the next.

As noted herein, a common FRET technology format utilizes TAQMAN® technology to detect the presence or absence of an amplification product, and hence, the presence or absence of PRRSV. The technology utilizes one single-stranded hybridization probe labeled with two moieties. When a first fluorescent moiety is excited with light of a suitable wavelength, the absorbed energy is transferred to a second quencher or acceptor moiety according to the principles of FRET. The second fluorescent moiety is preferably a quencher molecule. During the annealing step of the PCR reaction, the labeled hybridization probe binds to the target DNA (i.e., the amplification product) and is degraded by the 5′ to 3′ exonuclease activity of the Taq Polymerase during the subsequent elongation phase. After release, the excited fluorescent moiety and the quencher moiety become spatially separated from one another such that the emission from the first fluorescent moiety can be detected.

Another FRET technology format utilizes two hybridization probes. Each probe can be labeled with a different fluorescent moiety and the two probes are generally designed to hybridize in close proximity to each other in a target DNA molecule such as an amplification product. Efficient FRET can only take place when the fluorescent moieties are in direct local proximity (for example, within 5 nucleotides of each other as described herein) and when the emission spectrum of the donor fluorescent moiety overlaps with the absorption spectrum of the acceptor fluorescent moiety. The intensity of the emitted signal can be correlated with the number of original target DNA molecules (e.g., the number of PRRSV virions as reflected by the amount of PRRSV genetic material).

Yet another FRET technology format utilizes molecular beacon technology to detect the presence or absence of an amplification product, and hence, the presence or absence of PRRSV. Molecular beacon technology uses a hybridization probe labeled with a donor fluorescent moiety and an acceptor fluorescent moiety. The acceptor fluorescent moiety is generally a quencher, and the fluorescent labels (donor and acceptor) are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., the amplification products), the secondary structure of the probe is disrupted and the fluorescent moieties become separated from one another such that after excitation with light of a suitable wavelength, the emission of the first fluorescent moiety can be detected.

As an alternative to detection using FRET technology, an amplification product can be detected using a nucleic acid binding dye such as a fluorescent DNA binding dye. After interaction with the double-stranded nucleic acid, the nucleic acid bound dyes emit a fluorescence signal after excitation with light at a suitable wavelength. A nucleic acid intercalating dye may also be used. When nucleic acid binding dyes are used, a melting curve analysis is usually performed for confirmation of the presence of the amplification product.

Articles of Manufacture

The invention further provides for articles of manufacture to detect PRRSV. An article of manufacture according to the present invention can include primers and probes used to detect PRRSV, together with suitable packaging material. Preferably, the packaging includes a label or instructions for the use of the article in a method disclosed herein. Methods of designing primers and probes are disclosed herein, and representative examples of primers and probes that amplify and hybridize to PRRSV nucleic acid molecules are provided.

Having now generally described the invention, the same will be more readily understood through reference to the following examples which are provided by way of illustration, and are not intended to be limiting of the present invention, unless specified.

### EXAMPLES

The following examples are offered to illustrate, but not to limit the claimed invention.

### Example 1

### Materials and Methods

Two sets of type specific primers and a single fluorogenic probe were designed against a conserved region of the PRRSV genome. RNA was extracted from semen using the RNeasy kit and serum using Viral RNA Mini Kit, both from Qiagen (Valencia, Calif.) with the substitution of a modified viral lysis buffer. The assay was evaluated using cell-culture derived stock viruses representing North American, European-like and atypical PRRSV and Lelystad viruses, as well as clinical field samples from semen and blood. Specificity was evaluated by testing a panel of viruses that included PRRSV, several arteriviruses and other viruses that can produce similar clinical features as PRRSV. Assay sensitivity was measured by performing the test in parallel with a nested RT-PCR assay that had been previously validated against a swine bio-assay.

### Example 2

### Results

The RT-PCR assays both had 100% specificity based on the panel selected which included 12 European-like PRRSV from a diverse geographic area in the US, over 50 diverse U.S. PRRSV isolates, Lelystad virus, and several atypical isolates from Nebraska. The assay failed to amplify RNA extracted from other arteriviruses (EAV and LDV) as well as other non-related swine viruses (coronavirus, TGE, Circovirus, Pseudorabies, Parvo and SIV). The sensitivity of the North American PRRSV assay matched that of the nested RT-PCR with both North American and European-like PRRSV assays. The North American type assay detected up to a 10⁻⁸ dilution of a SDSU 23983 stock virus while the European-Like type assay detected up to a 10⁻⁷ dilution of Lelystad virus, both results matching the sensitivity of nested RT-PCR.

### Example 3

### Annotations of NC_{—}001961 (SEQ ID NO:1)

The 5′-UTR is positions 1 to 189. An ORF 1ab polyprotein is encoded by positions 190 to 12071. ORF2 is from positions 12073 to 12843 and it encodes a GP2 glycosylated envelope protein. ORF3 is from positions 12696 to 13460 and encodes GP3 envelope protein. ORF4 is from positions 13241 to 13777 and encodes gene product “GP4”. ORF5 is from positions 13788 to 14390 and encodes GP5 glycosylated envelope protein. ORF6 is from positions 14375 to 14899 and encodes membrane protein M. ORF7 is from positions 14889 to 15260 and encodes nucleocapsid protein N. The 3′-UTR is at positions 15261 to 15411. The polyadenylation site is at position 15412.

As would be evident to the skilled person in the art, the instant invention may be practiced with comparisons and reference to additional PRRSV genetic sequences which may be aligned with this sequence for comparison and identification of the various ORFs and the 3′-UTR.

### Example 4

### Additional Identifiers for the Sequences Provided in FIG. 2

Each sequence other than NC_{—}001961 in FIG. 2 is identified by a numeric indicator (followed by a nucleotide position indicator) at the left of each sequence in the alignment. Those indicators are provided below along with additional identifiers for those sequences as shown below, where “gb” stands for GenBank and the identifiers to the right of “gb” are GenBank accession numbers. The corresponding SEQ ID NO: in the instant application is also show.

| | |
|---|---|
| gi\|13377191\|gb\|AF299412.1\|AF299412, | SEQ ID NO: 2 |
| gi\|13096791\|gb\|AF066183.3\|AF066183, | SEQ ID NO: 3 |
| gi\|12240324\|gb\|AF331831.1\|AF331831, | SEQ ID NO: 4 |
| gi\|11192298\|gb\|U87392.3\|PRU87392, | SEQ ID NO: 5 |
| gi\|9931316\|gb\|AF159149.1\|AF159149, | SEQ ID NO: 6 |
| gi\|32441468\|gb\|AY256686.1, | SEQ ID NO: 7 |
| gi\|27549163\|gb\|AY150564.1, | SEQ ID NO: 8 |
| gi\|22658020\|gb\|AF176348.2, | SEQ ID NO: 9 |
| gi\|13377207\|gb\|AF299414.1\|AF299414, | SEQ ID NO: 10 |
| gi\|32441466\|gb\|AY256685.1, | SEQ ID NO: 11 |
| gi\|487429\|gb\|U03040.1\|PRU03040, | SEQ ID NO: 12 |
| gi\|4098187\|gb\|U75443.1\|PRU75443, | SEQ ID NO: 13 |
| gi\|7650192\|gb\|AF184212.1\|AF184212, | SEQ ID NO: 14 |
| gi\|2739142\|gb\|AF030306.1\|AF030306, | SEQ ID NO: 15 |
| gi\|1008891\|gb\|L39368.1\|PPSNE1A, | SEQ ID NO: 16 |
| gi\|12744849\|gb\|AF325691.1\|AF325691, | SEQ ID NO: 17 |
| gi\|725327\|gb\|L40898.1\|PPSORFS, | SEQ ID NO: 18 |
| gi\|437178\|gb\|U02095.1\|PRU02095, | SEQ ID NO: 19 |
| gi\|1008889\|gb\|L39366.1\|PPSMN1A, | SEQ ID NO: 20 |
| gi\|20271246\|gb\|AF494042.1, | SEQ ID NO: 21 |
| gi\|14250956\|gb\|AY032626.1, | SEQ ID NO: 22 |
| gi\|1008887\|gb\|L39364.1\|PPSKS1A, | SEQ ID NO: 23 |
| gi\|1008892\|gb\|L39369.1\|PPSSG1A, | SEQ ID NO: 24 |
| gi\|4886916\|gb\|AF121131.1\|AF121131, | SEQ ID NO: 25 |
| gi\|4680470\|gb\|AF066384.1\|AF066384, | SEQ ID NO: 26 |
| gi\|3746910\|gb\|AF090173.1\|AF090173, | SEQ ID NO: 27 |
| gi\|4512577\|gb\|AB023782.1, | SEQ ID NO: 28 |
| gi\|1089787\|dbj\|D45852.1, | SEQ ID NO: 29 |
| gi\|3097879\|gb\|U64929.1\|PRU64929, | SEQ ID NO: 30 |
| gi\|1008884\|gb\|L39361.1\|PPSIA1A, | SEQ ID NO: 31 |
| gi\|1008885\|gb\|L39362.1\|PPSIA6A, | SEQ ID NO: 32 |
| gi\|25361009\|gb\|AY150312.1, | SEQ ID NO: 33 |
| gi\|3097885\|gb\|U64932.1\|PRU64932, | SEQ ID NO: 34 |
| gi\|2739189\|gb\|AF035409.1\|AF035409, | SEQ ID NO: 35 |
| gi\|3097877\|gb\|U64928.1\|PRU64928, | SEQ ID NO: 36 |
| gi\|12711601\|gb\|AF317692.1\|AF317692, | SEQ ID NO: 37 |
| gi\|1008890\|gb\|L39367.1\|PPSMO1A, | SEQ ID NO: 38 |

### Example 5

### Detection of Additional North American PRRSV Isolates

To detect additional North American PRRSV isolates with the 3′-UTR sequences shown in FIG. 5 (isolates identified as 1269, 4202, and 18602), additional primers and probes were designed and used. Specifically, primers PRRSV-15302-F, PRRSV-15303-Fa, and PRRSV-15358-Ra, which differ from the corresponding sequence of SEQ ID NO:39 (a portion of SEQ ID NO:1), were designed for use to detect these additional isolates. The probe PRRSV-15332-MGB-1 was designed for use with these additional primers.

The use of these primers and probes, alone or in combination with other primers and probes of the invention, provide the capability to amplify genetic material from multiple North American isolates of PRRSV, including those with mutations relative to SEQ ID NOs:1 and 39.

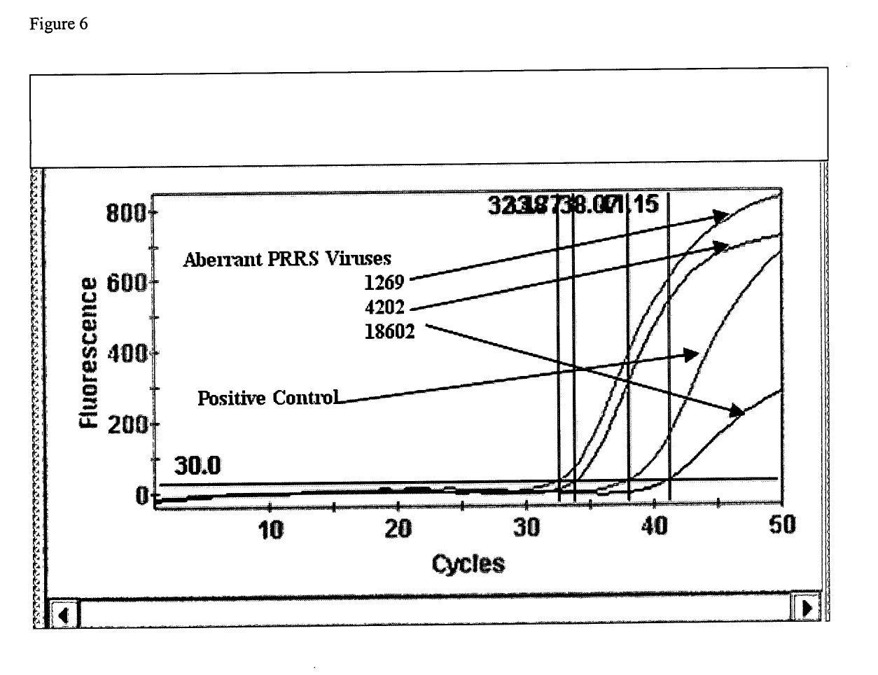

The results are shown in FIG. 6, wherein the primers and probes in FIG. 5 were used simultaneously in separate reactions containing material from PRSSV isolates 1269, 4202, and 18602 as well as a control PRSSV sequence. The combination was able to detect each of the three isolates as well as a control sample.

### Example 6

### Detection of Additional PRRSV in Europe and Western Asia

A panel of PRRSV collected from geographically diverse areas across the European continent are targeted by use of primers and probes based on sequences from ORF7 as shown in FIG. 7. The targeted region is positions 14640 to 14758 of the ORF7 region of Leylstad virus (GenBank accession number M96262 or M96262.2). Particularly preferred primers and probes for use in detecting PRRSV in Europe and Western Asia are Euro2-14646-F1, Euro2-14646-F2, or Euro2-14646-F3 as a forward primer; Euro2-14718-R1, Euro2-14718-R2, Euro2-14718-R3, or Euro2-14719-R as a reverse primer; and Euro2-14661-T, Euro2-14661-BHQ, or Euro2-14661-MGB as probes. Particularly preferred is the use of Euro2-14646-F3 as the forward primer, Euro2-14661-MGB as the probe, and Euro2-14718-R3 as the reverse primer.

These primers and probes are consistent with the following sequences of PRRSV. Stated differently, the above primers and probes may be used in the practice of the methods disclosed herein to detect PRRSV nucleic acids having the ORF7 region of the following sequences. The following indicators include additional identifiers such as “gb”, which stands for GenBank and the identifiers to the right of “gb” are GenBank accession numbers. The corresponding sequences are hereby incorporated by reference as if fully set forth.

gi|51094057|gb|AY588319.1| PRRSV LV4.2.1, complete genome
gi|30315358|gb|AF511526.1| PRRSV
gi|30315355|gb|AF511525.1| PRRSV
gi|1061205|emb|X92942.1|PRRSENVNP PRRSV
gi|2894348|emb|Z92708.1|PRVZ92708 PRRSV
gi|2894423|emb|Z92532.1|PRVZ92532 PRRSV
gi|2894421|emb|Z92531.1|PRVZ92531 PRRSV
gi|2894419|emb|Z92530.1|PRVZ92530 PRRSV
gi|2894415|emb|Z92528.1|PRVZ92528 PRRSV
gi|1369875|gb|L77924.1|PPSNO1ORFB PRRSV
gi|1369871|gb|L77922.1|PPSLE1ORFB PRRSV
gi|1369867|gb|L77920.1|PPSL2ORFB PRRSV
gi|1369859|gb|L77916.1|PPSH3ORFB PRRSV
gi|14486150|gb|AY035983.1| PRRSV
gi|14486148|gb|AY035982.1| PRRSV
gi|14486146|gb|AY035981.1| PRRSV
gi|14486144|gb|AY035980.1| PRRSV
gi|14486120|gb|AY035968.1| PRRSV
gi|14486118|gb|AY035967.1| PRRSV
gi|14486116|gb|AY035966.1| PRRSV
gi|14486114|gb|AY035965.1| PRRSV
gi|14486112|gb|AY035964.1| PRRSV
gi|14486110|gb|AY035963.1| PRRSV
gi|14486076|gb|AY035946.1| PRRSV
gi|14486074|gb|AY035945.1| PRRSV
gi|11934968|gb|AF297103.1|AF297103 PRRSV
gi|11934966|gb|AF297102.1|AF297102 PRRSV
gi|11934964|gb|AF297101.1|AF297101 PRRSV
gi|2894352|emb|Z92707.1|PRVZ92707 PRRSV
gi|2894350|emb|Z92706.1|PRVZ92706 PRRSV
gi|2894621|emb|Z92538.1|PRVZ92538 PRRSV
gi|2894625|emb|Z92537.1|PRVZ92537 PRRSV
gi|2894623|emb|Z92536.1|PRVZ92536 PRRSV
gi|2894429|emb|Z92535.1|PRVZ92535 PRRSV
gi|2894427|emb|Z92534.1|PRVZ92534 PRRSV
gi|2894425|emb|Z92533.1|PRVZ92533 PRRSV
gi|2894413|emb|Z92527.1|PRVZ92527 PRRSV
gi|2894411|emb|Z92526.1|PRVZ92526 PRRSV
gi|11125727|gb|M96262.2|LEYPOLYENV Lelystad virus
gi|294331|gb|L04493.1|PRWPOLGLYM PRRSV
gi|2894417|emb|Z92529.1|PRVZ92529 PRRSV
gi|1369879|gb|L77926.1|PPSNY4ORFB PRRSV
gi|1369863|gb|L77918.1|PPSHA1ORFB PRRSV
gi|1369855|gb|L77914.1|PPSBE1ORFB PRRSV
gi|14486106|gb|AY035961.1| PRRSV
gi|11934962|gb|AF297100.1|AF297100 PRRSV
gi|11934960|gb|AF297099.1|AF297099 PRRSV
gi|1369881|gb|L77927.1|PPSOX1ORFB PRRSV
gi|14486124|gb|AY035970.1| PRRSV
gi|14486122|gb|AY035969.1| PRRSV
gi|14486092|gb|AY035954.1| PRRSV
gi|39980583|gb|AY395081.1| PRRSV
gi|39980575|gb|AY395080.1| PRRSV
gi|39980567|gb|AY395079.1| PRRSV
gi|2695776|emb|AJ223078.1|PRR223078 PRRSV
gi|21303335|gb|AY035944.1| PRRSV
gi|14486100|gb|AY035958.1| PRRSV
gi|14486086|gb|AY035951.1| PRRSV
gi|14486080|gb|AY035948.1| PRRSV
gi|38146324|gb|AY366525.1| PRRSV
gi|21303323|gb|AY035941.1| PRRSV
gi|14486142|gb|AY035979.1| PRRSV
gi|14486140|gb|AY035978.1| PRRSV
gi|14486138|gb|AY035977.1| PRRSV
gi|14486134|gb|AY035975.1| PRRSV
gi|14486132|gb|AY035974.1| PRRSV
gi|14486084|gb|AY035950.1| PRRSV
gi|14486078|gb|AY035947.1| PRRSV
gi|11934970|gb|AF297104.1|AF297104 PRRSV
gi|30315361|gb|AF512378.1| PRRSV
gi|24637268|gb|AF438361.1| PRRSV
gi|14486108|gb|AY035962.1| PRRSV
gi|14486096|gb|AY035956.1| PRRSV
gi|14486094|gb|AY035955.1| PRRSV
gi|14486088|gb|AY035952.1| PRRSV
gi|14486082|gb|AY035949.1| PRRSV
gi|21303327|gb|AY035942.1| PRRSV
gi|14486128|gb|AY035972.1| PRRSV
gi|14486126|gb|AY035971.1| PRRSV
gi|14486098|gb|AY035957.1| PRRSV
gi|14486090|gb|AY035953.1| PRRSV 3
gi|24637266|gb|AF438360.1| PRRSV
gi|21303331|gb|AY035943.1| PRRSV
gi|14486104|gb|AY035960.1| PRRSV
gi|14486102|gb|AY035959.1| PRRSV
gi|1304593|gb|U40784.1|PRU40784 PRRSV
gi|1304591|gb|U40783.1|PRU40783 PRRSV
gi|1304589|gb|U40782.1|PRU40782 PRRSV
gi|1304585|gb|U40780.1|PRU40780 PRRSV
gi|1304581|gb|U40778.1|PRU40778 PRRSV
gi|1304575|gb|U40725.1|PRU40725 PRRSV
gi|1304573|gb|U40724.1|PRU40724 PRRSV
gi|1304569|gb|U40722.1|PRU40722 PRRSV
gi|1304587|gb|U40781.1|PRU40781 PRRSV
gi|1304583|gb|U40779.1|PRU40779 PRRSV
gi|1304579|gb|U40727.1|PRU40727 PRRSV
gi|1304577|gb|U40726.1|PRU40726 PRRSV
gi|1304571|gb|U40723.1|PRU40723 PRRSV
gi|14486130|gb|AY035973.1| PRRSV
gi|14486136|gb|AY035976.1| PRRSV

All references cited herein are hereby incorporated by reference in their entireties, whether previously specifically incorporated or not. As used herein, the terms “a”, “an”, and “any” are each intended to include both the singular and plural forms.

Having now fully described this invention, it will be appreciated by those skilled in the art that the same can be performed within a wide range of equivalent parameters, concentrations, and conditions without departing from the spirit and scope of the invention and without undue experimentation. While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method for detecting the presence or absence of PRRSV in a biological sample from an individual, such as a pig, said method comprising
performing real-time RT-PCR on said sample with at least a pair of oligonucleotide primers capable of producing an amplification product comprising a sequence from the 3′-UTR region of PRRSV
wherein said real-time RT-PCR comprises hybridization of said amplification product with one or more PRRSV probe, wherein said probe is labeled with a donor fluorescent moiety and a corresponding quencher or acceptor moiety; and
detecting the presence or absence of fluorescence over time from the RT-PCR reaction,
wherein the presence or absence of fluorescence is indicative of the presence or absence of PRRSV in said sample.

2. A method for detecting the presence or absence of PRRSV in a biological sample from an individual, such as a pig, said method comprising
performing real-time RT-PCR on said sample with at least a pair of oligonucleotide primers capable of producing an amplification product comprising a sequence from ORF 7 of PRRSV
wherein said real-time RT-PCR comprises hybridization of said amplification product with one or more PRRSV probe, wherein said probe is labeled with a donor fluorescent moiety and a corresponding quencher or acceptor moiety; and
detecting the presence or absence of fluorescence due to probe hydrolysis over time from the RT-PCR reaction,
wherein the presence or absence of fluorescence is indicative of the presence or absence of PRRSV in said sample.

3. The method of claim 1 wherein said pair of oligonucleotide primers comprise a forward primer selected from PRRSV-15308-F, PRRSV-153309-F3, PRRSV-153310-F4, PRRSV-153308-F5, PRRSV-153309-F6, LELY-14997-F, PRRSV-15302-F, PRRSV-15303-F, and PRRSV15303-Fa.

4. The method of claim 1 wherein said pair of oligonucleotide primers comprise a reverse primer selected from PRRSV-15409-R, PRRSV-15406-R, PRRSV-15360-R, PRRSV-15358-R, PRRSV-15357-R, PRRSV-15356-R, Lely-15093-R, and PRRSV-15360-Ra.

5. The method of claim 1 wherein said one or more PRRSV probe is selected from PRRSV-15336-T, Lelystad-15023-T, PRRSV-15344-MGB-1, PRRSV-15345-MGB-2, PRRSV-15337-MGB-3, PRRSV-15336-MGBR-4, PRRSV-15343-MGB-5, PRRSV-15343-MGBR-6, and PRRSV-15332-MGB-1.

6. The method of claim 1 wherein said pair of oligonucleotide primers and one or more PRRSV probe comprise PRRSV-15302-F, PRRSV-15303-F, PRRSV15303-Fa, PRRSV-15360-Ra, PRRSV-15358-R, and PRRSV-15344-MGB-1 and/or PRRSV-15332-MGB-1.

7. The method of claim 1 wherein said pair of oligonucleotide primers and one or more PRRSV probe comprise Lely-15005-F2, LELY-15074-R, and PRRSV-15344-MGB-1.

8. The method of claim 2 wherein said pair of oligonucleotide primers comprise a forward primer selected from Euro2-14646-F1, Euro2-14646-F2, and Euro2-14646-F3.

9. The method of claim 2 wherein said pair of oligonucleotide primers comprise a reverse primer selected from Euro2-14718-R1, Euro2-14718-R2, Euro2-14718-R3, and Euro2-14719-R.

10. The method of claim 2 wherein said one or more PRRSV probe is selected from Euro2-14661-T, Euro2-14661-BHQ, and Euro2-14661-MGB.

11. The method of claim 1 wherein said pair of oligonucleotide primers and one or more PRRSV probe comprise Euro2-14646-F3, Euro2-14661-MGB, and Euro2-14718-R3.

12. The method of claim 1 wherein said donor fluorescent moiety is selected from FAM or 6-FAM, fluorescein, HEX, TET, TAM, ROX, Cy3, Alexa, Texas Red fluorescein, Lucifer Yellow, B-pliycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimidyl 1-pyrenebutyrate, and 4-acetamido-4′-isothiocyanatostilbene-2,2′-disulfonic acid derivatives.

13. The method of claim 1 wherein said quencher or acceptor moiety is selected from MGB, TAMRA, BHQ (black hole quencher), LC™-RED 640 (LightCycler™-Red 640-N-hydroxysuccinimide ester), LC™-RED 705 (LightCycler™-Red 705-Phosphoramidite), cyanine dyes such as CY5 and CY5.5, lissamine rhodamine B sulfonyl chloride, tetramethyl rhodamine isothiocyanate, rhodamine x isothiocyanate, erythrosine isothiocyanate, fluorescein, and diethylenetriamine pentaacetate or other chelates of Lanthanide ions (e.g., Europium, or Terbium).

14. The method of claim 12 wherein said donor fluorescent moiety is FAM or 6-FAM.

15. The method of claim 13 wherein said quencher or acceptor moiety is MGB, TAMRA, or BHQ (black hole quencher).

16. The method of claim 1 wherein said sample is from an animal, such as a pig.

17. The method of claim 16 wherein said sample is selected from oral cavity swabs; cell containing tissues; necropsy tissues; punch biopsies; tonsil scrapings; bodily fluids such as semen, blood, or serum; and samples containing mononuclear cells of any kind or macrophages.

18. The method of claim 16 wherein said sample contain mononuclear cells or macrophages.

19. The method of claim 1 wherein said detecting is performed in real time.

20. The method of claim 1 wherein the presence of fluorescence after about 40 cycles of RT-PCR indicates the presence of PRRSV in said sample.

21. The method of claim 20 wherein the presence of fluorescence after about 30 cycles of RT-PCR indicates the presence of PRRSV in said sample.

22. The method of claim 21 wherein the presence of fluorescence after about 20 cycles of RT-PCR indicates the presence of PRRSV in said sample.

23. A kit comprising a pair of oligonucleotide primers capable of amplifying a sequence from the 3′-UTR or ORF7 of PRRSV and a probe labeled with a donor fluorescent moiety and a corresponding quencher or acceptor moiety, which probe hybridizes to an amplification product of said primers.
